(19) United States
(12) Patent Application Publication
(10) Pub. No.: US 2010/0273789 A1
(43) Pub. Date: Oct. 28, 2010

(54) AMINOACYL PRODRUGS

(75) Inventors: Hans-Georg Lerchen, Leverkusen (DE); Ursula Krenz, Leichlingen (DE); Michael Härter, Leverkusen (DE); Mark Jean Gnoth, Mettman (DE); Georges Von Degenfeld, Leverkusen (DE); Elke Dittrich-Wengenroth, Wuppertal (DE); Anja Buchmüller, Essen (DE); Susanne Röhrig, Hilden (DE); Swen Allerheiligen, Essen (DE); Elisabeth Perzborn, Wuppertal (DE); Christoph Gerdes, Koln (DE); Karl-Heinz Schlemmer, Wuppertal (DE); Metin Akbaba, Ratingen (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER SCHERING PHARMA AKTIENGESELLSCHAFT, Berlin (DE)

(21) Appl. No.: 12/668,619

(22) PCT Filed: Jun. 28, 2008
(86) PCT No.: PCT/EP08/05301

§ 371 Date: Jun. 28, 2010

(30) Foreign Application Priority Data
Jul. 11, 2007 (DE) 10 2007 032 347.8

(51) Int. Cl. A61K 31/5377; C07D 413/14; A61P 7/02

(52) U.S. Cl. 514/230.8; 544/139

(57) ABSTRACT

The present application relates to prodrug derivatives of 5-chloro-N-({(5S)-2-oxo-3-[2-fluoro-4-(3-oxomorpholin-4-yl)phenyl]-1,3-oxazolidin-5-yl}methyl)thiophene-2-carboxamide, processes for their preparation, their use for the treatment and/or prophylaxis of diseases, and their use for the manufacture of medicaments for the treatment and/or prophylaxis of diseases, especially of thromboembolic disorders.

The present application relates to prodrug derivatives of 5-chloro-N-({(5S)-2-oxo-3-[2-fluoro-4-(3-oxomorpholin-4-yl)phenyl]-1,3-oxazolidin-5-yl}methyl)thiophene-2-carboxamide, processes for their preparation, their use for the treatment and/or prophylaxis of diseases, and their use for the manufacture of medicaments for the treatment and/or prophylaxis of diseases, especially of thromboembolic disorders.

Prodrugs are derivatives of an active ingredient which undergo in vivo an enzymatic and/or chemical biotransformation in one or more stages before the actual active ingredient is liberated. A prodrug residue is ordinarily used in order to improve the profile of properties of the underlying active ingredient [P. Ettmayer et al., J. Med. Chem. 47, 2393 (2004)]. In order to achieve an optimal profile of effects it is necessary in this connection for the design of the prodrug residue as well as the desired mechanism of liberation to be coordinated very accurately with the individual active ingredient, the indication, the site of action and the administration route. A large number of medicaments is administered as prodrugs which exhibit an improved bioavailability by comparison with the underlying active ingredient, for example achieved by improving the physicochemical profile, specifically the solubility, the active or passive absorption properties or the tissue-specific distribution. An example which may be mentioned from the wide-ranging literature on prodrugs is: H. Bundgaard (Ed.), Design of Prodrugs: Bioreversible derivatives for various functional groups and chemical entities, Elsevier Science Publishers B.V., 1985.

5-Chloro-N-({(5S)-3-[2-fluoro-4-(3-oxomorpholin-4-yl)phenyl]-2-oxo-1,3-oxazolidin-5-yl}methyl)thiophene-2-carboxamide [compound (A)] is an orally effective, direct inhibitor of the serine protease factor Xa which performs an essential function in regulating the coagulation of blood. An oxazolidinone compound is currently undergoing in-depth clinical examination as a possible new active pharmaceutical ingredient for the prevention and therapy of thromboembolic disorders [S. Roehrig et al., J. Med. Chem. 48, 5900 (2005)].

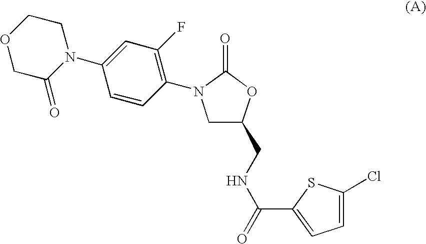

However, compound (A) has only a limited solubility in water and physiological media, making for example intravenous administration of the active ingredient difficult. It was therefore an object of the present invention to identify derivatives or prodrugs of compound (A) which have an improved solubility in the media mentioned and, at the same time, allow controlled liberation of the active ingredient (A) in the patient's body after administration.

WO 2005/028473 describes acyloxymethylcarbamate prodrugs of oxazolidinones which serve to increase the oral bioavailability. WO 01/00622 discloses acyl prodrugs of carbamate inhibitors of inosine-5′-monophosphate dehydrogenase. A further type of amide prodrugs for oxazolidinones which liberate the underlying active ingredient by a multistage activation mechanism is described in WO 03/006440.

The present invention relates to compounds of the general formula (I)

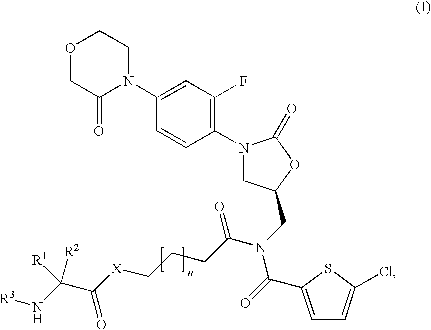

in which

n is the number 1 or 2,

X is an oxygen atom, sulfur atom or NH,

R¹ is the side group of a natural α-amino acid or its homologs or isomers,

R² is hydrogen or methyl,

R³ is hydrogen,

or

R¹ and R³ are linked via a (CH₂)₃ or (CH₂)₄ group and together with the nitrogen or carbon atom to which they are attached form a 5- or 6-membered ring, and the salts, solvates and solvates of the salts thereof.

Compounds according to the invention are the compounds of the formula (I) and the salts, solvates and solvates of the salts thereof, the compounds which are encompassed by formula (I) and are of the formulae mentioned hereinafter, and the salts, solvates and solvates of the salts thereof, and the compounds which are encompassed by formula (I) and are mentioned hereinafter as exemplary embodiments, and the salts, solvates and solvates of the salts thereof, insofar as the compounds encompassed by formula (I) and mentioned hereinafter are not already salts, solvates and solvates of the salts.

The compounds according to the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

Where the compounds according to the invention can occur in tautomeric forms, the present invention encompasses all tautomeric forms.

Salts preferred for the purposes of the present invention are physiologically acceptable salts of the compounds according to the invention. However, salts which are themselves unsuitable for pharmaceutical applications but can be used for example for isolating or purifying the compounds according to the invention are also encompassed.

Physiologically acceptable salts of the compounds according to the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Solvates refer for the purposes of the invention to those forms of the compounds according to the invention which form a complex in the solid or liquid state through coordination with solvent molecules. Hydrates are a specific form of solvates in which the coordination takes place with water. Solvates preferred in the context of the present invention are hydrates.

In the context of the present invention, the substituents have the following meaning unless otherwise specified:

The side group of an α-amino acid in the meaning of R¹ encompasses both the side groups of naturally occurring α-amino acids and the side groups of homologs and isomers of these α-amino acids. The α-amino acid may in this connection have both the L and the D configuration or else be a mixture of the L form and D form. Examples of side groups which may be mentioned are: hydrogen(glycine), methyl(alanine), propan-2-yl(valine), propan-1-yl(norvaline), 2-methylpropan-1-yl(leucine), 1-methylpropan-1-yl(isoleucine), butan-1-yl(norleucine), phenyl(2-phenylglycine), benzyl(phenylalanine), p-hydroxybenzyl(tyrosine), indol-3-ylmethyl(tryptophan), imidazol-4-ylmethyl(histidine), hydroxymethyl(serine), 2-hydroxyethyl(homoserine), 1-hydroxyethyl(threonine), mercaptomethyl(cysteine), methylthiomethyl(S-methylcysteine), 2-mercaptoethyl(homocysteine), 2-methylthioethyl(methionine), carbamoylmethyl(asparagine), 2-carbamoylethyl(glutamine), carboxymethyl(aspartic acid), 2-carboxyethyl(glutamic acid), 4-aminobutan-1-yl(lysine), 4-amino-3-hydroxybutan-1-yl(hydroxylysine), 3-aminopropan-1-yl(ornithine), 3-guanidinopropan-1-yl(arginine), 3-ureidopropan-1-yl(citrulline). Preferred α-amino acid side groups in the meaning of R² are hydrogen(glycine), methyl(alanine), propan-2-yl(valine), propan-1-yl(norvaline), imidazol-4-ylmethyl(histidine), hydroxymethyl(serine), 1-hydroxyethyl(threonine), carbamoylmethyl(asparagine), 2-carbamoylethyl(glutamine), 4-aminobutan-1-yl(lysine), 3-aminopropan-1-yl(ornithine), 3-guanidinopropan-1-yl(arginine). The L configuration is preferred.

If radicals in the compounds according to the invention are substituted, the radicals may, unless otherwise specified, be substituted one or more times. In the context of the present invention, all radicals which occur more than once have a mutually independent meaning. Substitution by one or two identical or different substituents is preferred. Substitution by one substituent is very particularly preferred.

Preference is given to compounds of the formula (I) in which

n is the number 1 or 2,

X is an oxygen atom, sulfur atom or NH,

R¹ is hydrogen, methyl, propan-2-yl, propan-1-yl, 2-methylpropan-1-yl, imidazol-4-ylmethyl, hydroxymethyl, 1-hydroxyethyl, carboxymethyl, 2-carboxyethyl, carbamoylmethyl, 2-carbamoylethyl, 4-aminobutan-1-yl, 3-aminopropan-1-yl or 3-guanidinopropan-1-yl, benzyl or 4-hydroxybenzyl,

R² is hydrogen or methyl,

R³ is hydrogen,

or

R¹ and R³ are linked via a (CH₂)₃ or (CH₂)₄ group and together with the nitrogen or carbon atom to which they are attached form a 5- or 6-membered ring, and the salts, solvates and solvates of the salts thereof.

Preference is also given to compounds of the formula (I) in which

n is the number 1 or 2,

X is NH,

R¹ is hydrogen, methyl, propan-2-yl, 2-methylpropan-1-yl, imidazol-4-ylmethyl, hydroxymethyl, 1-hydroxyethyl, carboxymethyl, 2-carboxyethyl, carbamoyl methyl, 2-carbamoylethyl, 4-aminobutan-1-yl, benzyl or 4-hydroxybenzyl,

R² is hydrogen,

R³ is hydrogen,

and the salts, solvates and solvates of the salts thereof.

Preference is also given to compounds of the formula (I) in which n is the number 2.

Preference is also given to compounds of the formula (I) in which X is NH.

Preference is also given to compounds of the formula (I) in which R¹ is hydrogen, methyl, propan-2-yl, 2-methylpropan-1-yl, imidazol-4-ylmethyl, hydroxymethyl, 1-hydroxyethyl, carboxymethyl, 2-carboxyethyl, carbamoylmethyl, 2-carbamoylethyl, 4-aminobutan-1-yl, 3-guanidinopropan-1-yl, benzyl or 4-hydroxybenzyl.

Preference is also given to compounds of the formula (I), in which R¹ is hydrogen, methyl, propan-2-yl, 2-methylpropan-1-yl, imidazol-4-ylmethyl, hydroxymethyl, 1-hydroxyethyl, carboxymethyl, 2-carboxyethyl, carbamoylmethyl, 2-carbamoylethyl, 4-aminobutan-1-yl, benzyl or 4-hydroxybenzyl.

Preference is also given to compounds of the formula (I), in which R² is hydrogen.

Preference is also given to compounds of the formula (I), in which R³ is hydrogen.

The invention further relates to a process for preparing the compounds of the formula (I), characterized in that either

[A] the compound of the formula

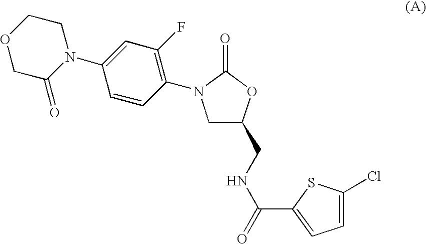

is initially converted in an inert solvent in the presence of a base with a compound of the formula

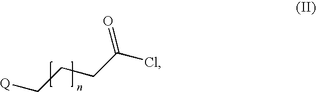

in which n has the meaning indicated above,

and

Q is a leaving group such as, for example, chlorine, bromine or iodine, into a compound of the formula

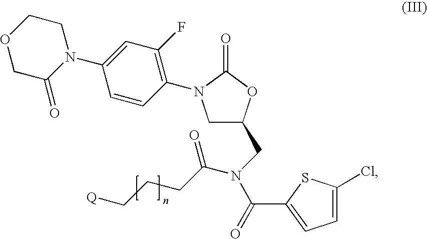

in which n and Q have the meaning indicated above,

the latter is then reacted according to the process

[A1] in an inert solvent with the cesium salt of an α-aminocarboxylic acid or an α-aminothiocarboxylic acid of the formula

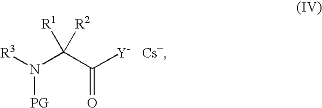

in which R¹, R² and R³ have the meaning indicated above,

PG is an amino protective group such as, for example, tert-butoxycarbonyl (Boc) or benzyloxycarbonyl (Z),

and

Y is O or S,

to give a compound of the formula

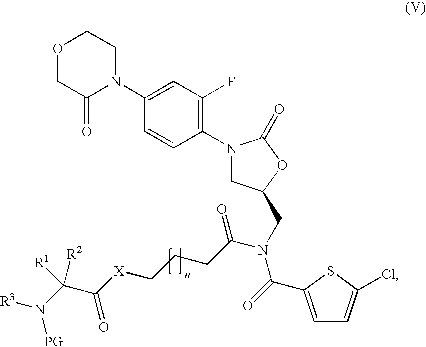

in which n, R¹, R², R³ and PG have the meaning indicated above, and

X is O or S,

and subsequently the protective group PG is removed by conventional methods to result in a compound of the formula

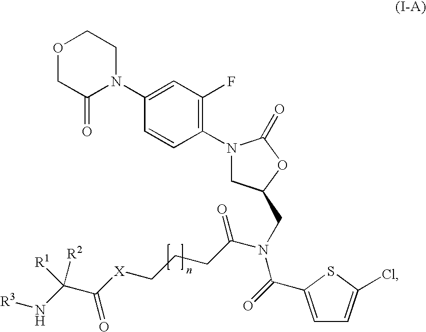

in which n, R¹, R² and R³ have the meaning indicated above, and

X is O or S,

or

[A2] is reacted in an inert solvent in the presence of a base with an α-aminothiocarboxylic acid of the formula

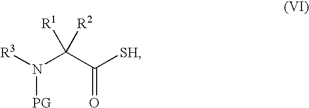

in which R¹, R² and R³ have the meaning indicated above,

PG is an amino protective group, such as, for example, tert-butoxycarbonyl (Boc) or benzyloxycarbonyl (Z),

to give a compound of the formula

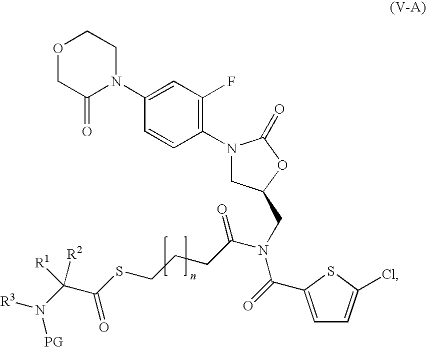

in which n, R¹, R², R³ and PG have the meaning indicated above, and

subsequently the protective group PG is removed by conventional methods to result in a compound of the formula

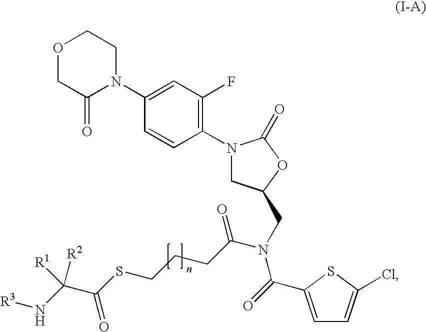

in which n, R¹, R² and R³ have the meaning indicated above,

or

[B] compound (A) is reacted in an inert solvent in the presence of a base with a compound of the formula

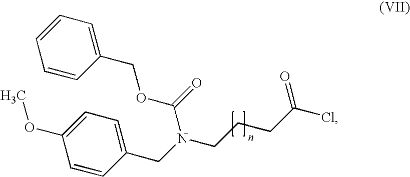

in which n has the meaning indicated above,

to give a compound of the formula

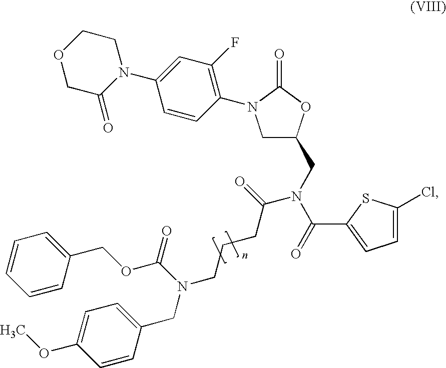

in which n has the meaning indicated above,

subsequently the protective groups are removed by conventional methods to result in a compound of the formula

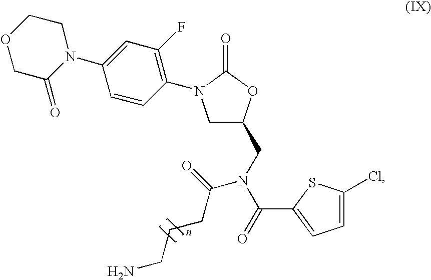

in which n has the meaning indicated above, and then, in the presence of a base, reacted with a compound of the formula

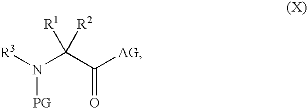

in which R¹, R² and R³ have the meaning indicated above,

AG is hydroxyl or halogen, preferably chlorine or bromine, or together with the carbonyl group form an activated ester, preferably an N-hydroxysuccinimide ester, or a mixed anhydride, preferably an alkyl formate, particularly preferably an ethyl formate, and

PG is an amino protective group, such as, for example, tert-butoxycarbonyl (Boc) or benzyloxycarbonyl (Z),

to give a compound of the formula

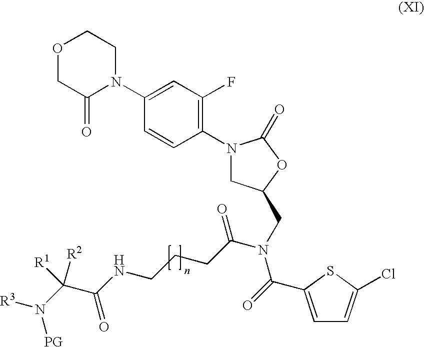

in which n, R¹, R², R³ and PG have the meaning indicated above,

and subsequently the protective group PG is removed by conventional methods to result in a compound of the formula

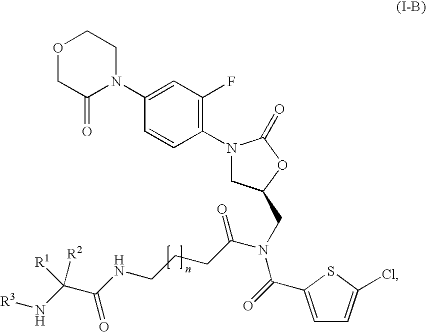

in which n, R¹, R² and R³ have the meaning indicated above,

and the compounds of the formula (I-A) or (I-B) resulting in each case are converted where appropriate with the appropriate (i) solvents and/or (ii) acids into the solvates, salts and/or solvates of the salts thereof.

The compounds of the formulae (I-A), (I-B) and (IX) can also be present in the form of their salts. These salts can be converted where appropriate by treatment with the appropriate (i) solvents and/or (ii) bases into the free base.

Functional groups present where appropriate in the radical R¹ may, if expedient or necessary, also be in temporarily protected form in the reaction sequences described above. The introduction and removal of such protective groups, as well as of the protective groups PG, takes place in this connection by conventional methods known from peptide chemistry [see, for example, T. W. Greene and P. G. M. Wuts, Protective Groups in Organic Synthesis, Wiley, New York, 1999; M. Bodanszky and A. Bodanszky, The Practice of Peptide Synthesis, Springer-Verlag, Berlin, 1984].

Such protective groups which are present where appropriate in R¹ may in this connection be removed at the same time as the elimination of PG or in a separate reaction step before or after the elimination of PG.

The amino protective group PG preferably used in the above processes is tert-butoxycarbonyl (Boc) or benzyloxycarbonyl (Z). Elimination of these protective groups and elimination of the protective groups in process step (VIII)→(X) is carried out by conventional methods, preferably by reacting with a strong acid such as hydrogen chloride, hydrogen bromide or trifluoroacetic acid in an inert solvent such as dioxane, dichloromethane or acetic acid.

The inert solvents preferably used in process steps (A)+(II)→(III) and (A)+(VII)→(VIII) are tetrahydrofuran, N,N-dimethylformamide or dimethyl sulfoxide; N,N-dimethylformamide is particularly preferred. A particularly suitable base in these reactions is sodium hydride. The reactions mentioned are generally carried out in a temperature range from 0° C. to +40° C. under atmospheric pressure.

The inert solvents preferably used in process steps (III)+(VI)→(V-A) and (IX)+(X)→(XI) are tetrahydrofuran, N,N-dimethylformamide or dimethyl sulfoxide; N,N-dimethylformamide is particularly preferred. A particularly suitable base in these reactions is ethyldiisopropylamine. The reactions mentioned are generally carried out in a temperature range from 0° C. to +40° C. under atmospheric pressure.

Process step (III)+(IV)→(V) preferably takes place in N,N-dimethylformamide as solvent. The reaction is generally carried out in a temperature range from 0° C. to +50° C., preferably at +20° C. to +50° C., under atmospheric pressure. The reaction can also be carried out advantageously with ultrasound treatment.

The compounds of the formulae (II), (IV), (VI), (VII) and (X) are commercially available, known from the literature or can be prepared by processes customary in the literature. Preparation of compound (A) is described in the Examples.

Preparation of the compounds according to the invention can be illustrated by the following synthesis scheme:

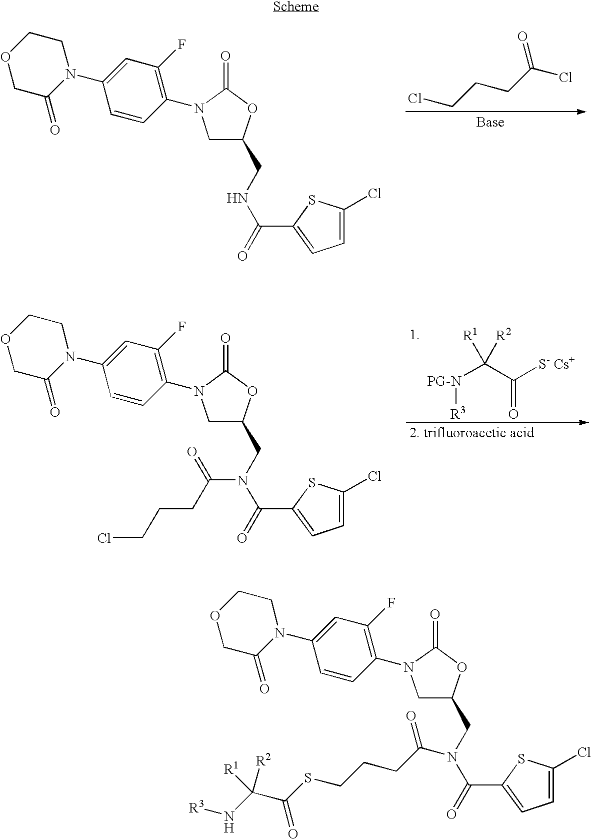

The compounds according to the invention and their salts represent useful prodrugs of the active ingredient compound (A). On the one hand, they show good stability for example at pH 4 and, on the other hand, they show efficient conversion into the active ingredient compound (A) in vivo. The compounds according to the invention moreover have good solubility in water and other physiologically tolerated media, making them suitable for therapeutic use especially on intravenous administration.

The present invention further relates to the use of the compounds according to the invention for the treatment and/or prophylaxis of disorders, preferably of thromboembolic disorders and/or thromboembolic complications.

The “thromboembolic disorders” include in the context of the present invention in particular disorders such as myocardial infarction with ST segment elevation (STEMI) and without ST segment elevation (non-STEMI), stable angina pectoris, unstable angina pectoris, reocclusions and restenoses following coronary interventions such as angioplasty or aortocoronary bypass, peripheral arterial occlusive diseases, pulmonary embolisms, deep venous thromboses and renal vein thromboses, transient ischemic attacks, and thrombotic and thromboembolic stroke.

The substances are therefore also suitable for the prevention and treatment of cardiogenic thromboembolisms, such as, for example, cerebral ischemias, stroke and systemic thromoboembolism and ischemias, in patients with acute, intermittent or persistent cardiac arrhythmias such as, for example, atrial fibrillation, and those undergoing cardioversion, also in patients with heart valve diseases or with artificial heart valves. The compounds according to the invention are additionally suitable for the treatment of disseminated intravascular coagulation (DIC).

Thromboembolic complications also occur in association with microangiopathic hemolytic anemia, extracorporeal circulations, such as hemodialysis, and heart valve prostheses.

The compounds according to the invention are additionally suitable also for the prophylaxis and/or treatment of atherosclerotic vascular disorders and inflammatory disorders such as rheumatic disorders of the musculoskeletal system, furthermore likewise for the prophylaxis and/or treatment of Alzheimer's disease. The compounds according to the invention can additionally be employed for inhibiting tumor growth and metastasis formation, for microangiopathies, age-related macular degeneration, diabetic retinopathy, diabetic nephropathy and other microvascular disorders, and for the prevention and treatment of thromoembolic complications such as, for example, venous thromboembolisms in tumor patients, especially those undergoing major surgical procedures or chemotherapy or radiotheraphy.

The present invention further relates to the use of the compounds according to the invention for the treatment and/or prophylaxis of disorders, especially of the aforementioned disorders.

The present invention further relates to the use of the compounds according to the invention for the manufacture of a medicament for the treatment and/or prophylaxis of disorders, especially of the aforementioned disorders.

The present invention further relates to a method for the treatment and/or prophylaxis of disorders, especially of the aforementioned disorders, using the compounds according to the invention.

The present invention further relates to medicaments comprising a compound according to the invention and one or more further active ingredients, especially for the treatment and/or prophylaxis of the aforementioned disorders. Examples of suitable combination active ingredients which may preferably be mentioned are:
lipid-lowering agents, especially HMG-CoA (3-hydroxy-3-methylglutarylcoenzyme A) reductase inhibitors;
coronary therapeutics/vasodilators, especially ACE (angiotensin converting enzyme) inhibitors, All (angiotensin II) receptor antagonists; β-adrenoceptor antagonists; alpha-1 adrenoceptor antagonists; diuretics; calcium channel blockers; substances which bring about an increase in cyclic guanosine monophosphate (cGMP), such as, for example, stimulators of soluble guanylate cyclase;
plasminogen activators (thrombolytics/fibrinolytics) and compounds which increase thrombolysis/fibrinolysis, such as inhibitors of plasminogen activator inhibitor (PAI inhibitors) or inhibitors of the thrombin-activated fibrinolysis inhibitor (TAFI inhibitors);
substances having anticoagulant activity (anticoagulants);
platelet aggregation-inhibiting substances (platelet aggregation inhibitors);
fibrinogen receptor antagonists (glycoprotein IIb/IIIa antagonists);
and antiarrhythmics.

The present invention further relates to medicaments which comprise at least one compound according to the invention, normally together with one or more inert, non-toxic, pharmaceutically suitable excipients, and to the use thereof for the aforementioned purposes.

The compounds according to the invention can act systemically and/or locally. For this purpose, they can be administered in a suitable way such as, for example, by the oral, parenteral, pulmonary or nasal route. The compounds according to the invention can be administered in administration forms suitable for these administration routes.

Suitable for oral administration are administration forms which function according to the prior art and deliver the compounds according to the invention rapidly and/or in modified fashion, and which contain the compounds according to the invention in crystalline and/or amorphized and/or dissolved form, such as, for example, tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates or sterile powders.

Suitable for the other administration routes are, for example, pharmaceutical forms for inhalation, such as power inhalers or nebulizers, or pharmaceutical forms which can be administered nasally, such as drops, solutions or sprays.

Parenteral administration is preferred, especially intravenous administration.

The compounds according to the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colorants (e.g. inorganic pigments such as, for example, iron oxides) and masking flavors and/or odors.

It has generally proved advantageous to administer on parenteral administration amounts of about 0.001 to 1 mg/kg, preferably about 0.01 to 0.5 mg/kg, of body weight to achieve effective results, and on oral administration the dosage is about 0.01 to 100 mg/kg, preferably about 0.01 to 20 mg/kg, and very particularly preferably 0.1 to 10 mg/kg, of body weight.

It may nevertheless be necessary where appropriate to deviate from the stated amounts, in particular as a function of the body weight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data for the liquid/liquid solutions are in each case based on volume.

### A. EXAMPLES

### Abbreviations and Acronyms:

abs. absolute

Boc tert-butoxycarbonyl

DMF N,N-dimethylformamide

DMSO dimethyl sulfoxide

h hour(s)

HPLC high pressure, high performance liquid chromatography

LC-MS coupled liquid chromatography-mass spectrometry

min minute(s)

MS mass spectrometry

NMR nuclear magnetic resonance spectrometry

Pd/C palladium on activated carbon

quant. quantitative (for yield)

RT room temperature

Rₜ retention time (for HPLC)

UV ultraviolet spectrometry

v/v volume to volume ratio (of a solution)

Z benzyloxycarbonyl

LC-MS and HPLC methods:

Method 1: Instrument: HP 1100 with DAD detection; column: Kromasil 100 RP-18, 60 mm×2.1 mm, 3.5 μm; mobile phase A: 5 ml of perchloric acid (70% strength)/l of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

Method 2: Instrument: HP 1100 with DAD detection; column: Kromasil 100 RP-18, 60 mm×2.1 mm, 3.5 μm; mobile phase A: 5 ml of perchloric acid (70% strength)/l of water, mobile phase B: acetonitrile; gradient: 0 min 2% B→0.5 min 2% B→4.5 min 90% B→9 min 0% B→9.2 min 2% B→10 min 2% B; flow rate: 0.75 ml/min; column temperature: 30° C.; UV detection: 210 nm.

Method 3: MS instrument type: Micromass ZQ; HPLC instrument type: HP 1100 Series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 4: Instrument: Micromass GCT, GC6890; column: Restek RTX-35MS, 30 m×250 μm×0.25 μm; constant helium flow rate: 0.88 ml/min; oven: 60° C.; inlet: 250° C.; gradient: 60° C. (maintained for 0.30 min), 50° C/min→120° C., 16° C/min→250° C., 30° C/min→300° C. (maintained for 1.7 min).

Method 5: column: GROM-SIL 120 ODS-4 HE, 10 μM, 250 mm×30 mm; flow rate: 50 ml/min; mobile phase and gradient program: acetonitrile/0.1% aqueous formic acid 10:90 (0-3 min), acetonitrile/0.1% aqueous formic acid 10:90→95:5 (3-27 min), acetonitrile/0.1% aqueous formic acid 95:5 (27-34 min), acetonitrile/0.1% aqueous formic acid 10:90 (34-38 min); temperature: 22° C.; UV detection: 254 nm.

Method 6: Instrument: Micromass ZQ; HPLC instrument type: HP 1100 Series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min. 2 ml/min; temperature: 50° C.; UV detection: 210 nm.

Method 7 (LC-MS): MS instrument type: Waters (Micromass) Quattro Micro; HPLC instrument type: Agilent 1100 Series; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A→4.01 min 100% A (flow rate 2.5 ml)→5.00 min 100% A; oven: 50° C.; flow rate: 2 ml/min; UV detection: 210 nm.

Method 8 (LC-MS): MS instrument type: Micromass ZQ; HPLC instrument type: Waters Alliance 2795; column: Phenomenex Synergi 2.5μ MAX-RP 100A Mercury 20 mm×4 mm; mobile phase A: 1 l of water +0.5 ml of 50% strength formic acid, mobile phase B: 1 l of acetonitrile +0.5 ml of 50% strength formic acid; gradient: 0.0 min 90% A→0.1 min 90% A→3.0 min 5% A→4.0 min 5% A→4.01 min 90% A; flow rate: 2 ml/min;; oven: 50° C.; UV detection: 210 nm.

Method 9 (analytical HPLC): Instrument: HP1090 Series II; column: Waters XTerra 018-5, 3.9 mm×150 mm WAT 186000478; mobile phase A: 10 ml of 70% strength perchloric acid in 2.5 liter of water, mobile phase B: acetonitrile; gradient: 0.0 min 20% B→1 min 20% B→4 min 90% B→6 min 90% B→8 min 20% B. temperature: 40° C.; flow rate: 1 ml/min.

Method 10 (analytical HPLC): Instrument: HP 1090 Series II; column: Merck Chromolith Speed ROD RP-18e, 50 mm×4.6 mm; precolumn Chromolith Guard Cartridge Kit, RP-18e, 5-4.6 mm; mobile phase A: 5 ml of perchloric acid (70% strength)/l of water, mobile phase B: acetonitrile; gradient: 0 min 20% B→0.5 min 20% B→3 min 90% B→3.5 min 90% B→3.51 min 20% B→4 min 20% B; flow rate: 5 ml/min; column temperature: 40° C.; UV detection: 210 nm.

Method 11 (preparative HPLC): Instrument: Gilson with UV detector, column: Kromasil C18, 5 μm/250 mm×20 mm (flow rate: 25 ml/min); mobile phase A: water (0.01% trifluoroacetic acid), mobile phase B: acetonitrile (0.01% trifluoroacetic acid); gradient: 0 min 5-20% B, 10 min-15 min 5-20% B, 45 min 90% B, 50 min 90% B; UV detection: 210 nm; flow rate: 25 ml/min.

Method 12 (preparative HPLC): Instrument: Gilson with UV detector, column: YMC ODS AQ C18, 10 μm/250 mm×30 mm (flow rate: 50 ml/min); mobile phase A: water (0.01% trifluoroacetic acid), mobile phase B: acetonitrile (0.01% trifluoroacetic acid); gradient: 0 min 5-20% B, 10 min-15 min 5-20% B, 45 min 90% B, 50 min 90% B; UV detection: 210 nm; flow rate: 50 ml/min.

Method 13 (LC-MS): Instrument: Micromass Quattro Premier with Waters UPLC Acquity; column: Thermo Hypersil GOLD 1.9μ, 50 mm×1 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→1.5 min 10% A→2.2 min 10% A; oven: 50° C.; flow rate: 0.33 ml/min; UV detection: 210 nm.

NMR Spectrometry:

NMR measurements were carried out at a proton frequency of 400.13 MHz or 500.13 MHz. The samples were normally dissolved in DMSO-d₆; temperature: 302 K.

Starting Compounds:

The starting material used was 5-chloro-N-({(5S)-2-oxo-3-[2-fluoro-4-(3-oxomorpholin-4-yl)phenyl]-1,3-oxazolidin-5-yl}methyl)thiophene-2-carboxamide [compound (A)].

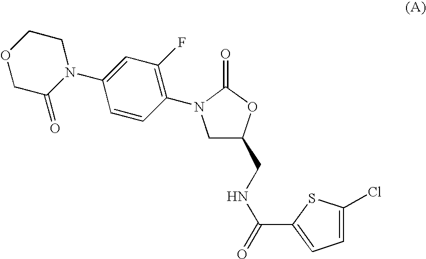

### Example 1A

### 5-Chlorothiophene-2-carbonyl chloride

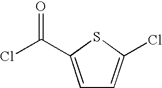

137 ml (1.57 mol) of oxalyl chloride were added to a suspension of 51.2 g (0.315 mmol) of 5-chlorothiophene-2-carboxylic acid in 307 ml of dichloromethane. After addition of 2 drops of DMF the mixture was stirred at room temperature for 15 hours. The solvent and excess oxalyl chloride were then removed on a rotary evaporator. The residue was distilled under reduced pressure. The product boiled at 74-78° C. and a pressure of 4-5 mbar. This gave 50.5 g (87% of theory) of an oil which solidified on storage in the fridge.

¹H-NMR (400 MHz, CDCl₃, δ/ppm): 7.79 (d, 1H), 7.03 (d, 1H).

GC/MS (Method 4): Rₜ=5.18 min.

MS (EI+, m/z): 180/182/184 (2³⁵Cl/³⁷Cl) M⁺.

### Example 2A

### ((S)-2,3-Dihydroxypropyl)-5-chlorothiophene-2-carboxamide

(from: C. R. Thomas, Bayer HealthCare AG, DE-10300111-A1 (2004).)

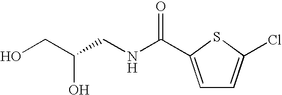

At 13-15° C., 461 g (4.35 mol) of sodium bicarbonate and 350 g (3.85 mol) of (2S)-3-aminopropane-1,2-diol hydrochloride were initially charged in 2.1 l of water, and 950 ml of 2-methyltetrahydrofuran were added. With cooling at 15-18° C., 535 g (2.95 mol) of 5-chlorothiophene-2-carbonyl chloride (compound from Example 1A) in 180 ml of toluene were added dropwise to this mixture over a period of two hours. For work-up, the phases were separated and a total of 1.5 l of toluene was added in a plurality of steps to the organic phase. The precipitated product was filtered off with suction, washed with ethyl acetate and dried. This gave 593.8 g (92% of theory) of product.

### Example 3A

### ((S)-3-Bromo-2-hydroxypropyl)-5-chlorothiophene-2-carboxamide

(from: C. R. Thomas, Bayer HealthCare AG, DE-10300111-A1 (2004).)

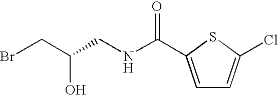

Over a period of 30 minutes, 301.7 ml of a 33% strength solution of hydrogen bromide in acetic acid were, at 21-26° C., added to a suspension of 100 g (0.423 mol) of the compound from Example 2A in 250 ml of glacial acetic acid. 40 ml of acetic anhydride were then added, and the reaction mixture was stirred at 60-65° C. for three hours. At 20-25° C., 960 ml of methanol were then added over a period of 30 minutes. The reaction mixture was stirred under reflux for 2.5 hours and then at 20-25° C. overnight. For work-up, the solvents were distilled off under reduced pressure at about 95 mbar. 50 ml of n-butanol and 350 ml of water were added to the suspension that remained. The precipitated product was filtered off with suction, washed with water and dried. This gave 89.8 g (71% of theory) of product.

### Example 4A

### 5-Chloro-N-[(2S)-oxiran-2-ylmethyl]thiophene-2-carboxamide

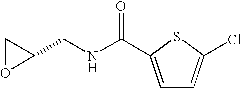

155 g (1.12 mol) of powdered potassium carbonate were added to a solution of 50 g (0.167 mol) of the compound from Example 3A in 500 ml of anhydrous THF, and the mixture was stirred at room temperature for 3 days. The inorganic salts were then filtered off with suction over a layer of kieselguhr and washed twice with in each case 100 ml of THF, and the filtrate was concentrated on a rotary evaporator at room temperature. This gave 36 g (81% of theory) of product.

¹H-NMR (400 MHz, DMSO-d₆, δ/ppm): 8.81 (t, 1H), 7.68 (d, 1H), 7.19 (d, 1H), 3.55-3.48 (m, 1H), 3.29-3.22 (m, 1H), 3.10-3.06 (m, 1H), 2.75-2.72 (m, 1H), 2.57-2.54 (m, 1H).

HPLC (Method 1): Rₜ=3.52 min.

MS (DCl, NH₃, m/z): (³⁵Cl/³⁷Cl) 218/220 (M+H)⁺, 235/237 (M+NH₄)⁺.

### Example 5A

### N,N-Dibenzyl-2-fluoro-4-iodoaniline

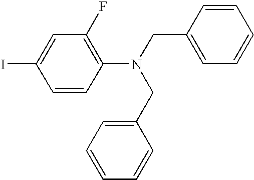

In a mixture of 100 ml of water and 200 ml of dichloromethane, 24.37 g (0.103 mol) of 2-fluoro-4-iodoaniline, 31.8 ml (0.267 mol) of benzyl bromide, 23.98 g (0.226 mol) of sodium carbonate and 1.9 g (5.14 mmol) of tetra-n-butylammonium iodide were heated at reflux for six days. After cooling to room temperature, the phases were separated from one another. The organic phase was washed with water and saturated sodium chloride solution and dried over anhydrous sodium sulfate. After filtration, the solvent was removed on a rotary evaporator. The residue obtained was purified by filtration with suction through silica gel using the mobile phase cyclohexane. This gave 35 g (82% of theory) of the title compound.

¹H-NMR (400 MHz, DMSO-d₆, δ/ppm): 7.48 (1H, dd), 7.32-7.21 (m, 11H), 6.69 (dd, 1 H), 4.33 (s, 4H).

HPLC (Method 1): Rₜ=5.87 min.

MS (DCl, NH₃, m/z): 418 (M+H)⁺.

### Example 6A

### 4-[4-(Dibenzylamino)-3-fluorophenyl]morpholin-3-one

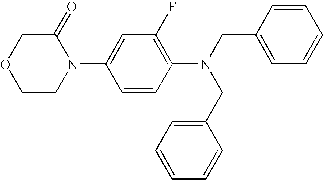

1.5 g (3.59 mmol) of the compound from Example 5A were dissolved in 20 ml of anhydrous dioxane, and 0.45 g (4.49 mmol) of morpholinone, 137 mg (0.719 mmol) of copper(I) iodide, 1.53 g (7.19 mmol) of potassium phosphate and 153 μl (1.44 mmol) of N,N′-dimethylethylenediamine were added in succession. The reflux apparatus was inertized by repeated application of a slightly reduced pressure and venting with argon. The reaction mixture was heated at reflux for 15 hours. After this period of time, the mixture was allowed to cool to room temperature. Water was added, and the mixture was extracted with ethyl acetate. The organic extract was washed successively with water and saturated sodium chloride solution. The extract was dried over anhydrous magnesium sulfate and then filtered, and the filtrate was freed from the solvent under reduced pressure. The residue was purified by filtration with suction through silica gel using the mobile phase cyclohexane/ethyl acetate 1:1. This gave 1.38 g (98% of theory) of the title compound.

¹H-NMR (400 MHz, DMSO-d₆, δ/ppm): 7.32-7.28 (m, 9H), 7.26-7.20 (m, 2H), 7.00-6.92 (m, 2H), 4.33 (s, 4H), 4.15 (s, 2H), 3.91 (dd, 2H), 3.55 (dd, 2H).

HPLC (Method 1): Rₜ=4.78 min.

MS (DCl, NH₃, m/z): 391 (M+H)⁺.

### Example 7A

### 4-(4-Amino-3-fluorophenyl)morpholin-3-one

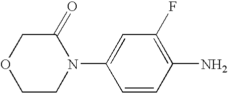

Method 1:

700 mg (1.79 mmol) of the compound from Example 6A were dissolved in 70 ml of ethanol, and 95 mg of palladium on activated carbon (10%) were added. The mixture was hydrogenated at room temperature and a hydrogen pressure of 1 bar for one hour. The catalyst was then filtered off through a little kieselguhr and the filtrate was concentrated on a rotary evaporator. This gave 378 mg (95% of theory) of the title compound.

¹H-NMR (400 MHz, DMSO-d₆, δ/ppm): 7.04 (dd, 1H), 6.87 (dd, 1H), 6.73 (dd, 1H), 5.17 (s, broad, 2H), 4.12 (s, 2H), 3.91 (dd, 2H), 3.62 (dd, 2H).

HPLC (Method 1): Rₜ=0.93 min.

MS (DCl, NH₃, m/z): 211 (M+H)⁺, 228 (M+NH₄)⁺.

Method 2:

Under argon, a suspension of 29.6 g (125 mmol) of 2-fluoro-4-iodoaniline, 15.8 g (156 mmol, 1.25 eq.) of morpholin-3-one [J.-M. Lehn, F. Montavon, Helv. Chim. Acta 1976, 59, 1566-1583], 9.5 g (50 mmol, 0.4 eq.) of copper(I) iodide, 53.1 g (250 mmol, 2 eq.) of potassium phosphate and 8.0 ml (75 mmol, 0.6 eq.) of N,N′-dimethylethylenediamine in 300 ml of dioxane was stirred under reflux overnight. After cooling to RT, the reaction mixture was filtered through a layer of kieselguhr and the residue was washed with dioxane. The combined filtrates were concentrated under reduced pressure. The crude product was purified by flash chromatography (silica gel 60, dichloromethane/methanol 100:1→100:3). This gave 24 g (74% of theory) of the title compound.

LC-MS (Method 3): Rₜ=0.87 min;

MS (ESIpos): m/z=211 [M+H]⁺;

¹H-NMR (500 MHz, DMSO-d₆): δ=7.05 (dd, 1H), 6.87 (dd, 1H), 6.74 (dd, 1H), 5.14 (s, 2H), 4.11 (s, 2H), 3.92 (dd, 2H), 3.63 (dd, 2H).

### Example 8A

### 5-Chloro-N-[2R)-3-{[2-fluoro-4-(3-oxomorpholin-4-yl)phenyl]amino}-2-hydroxypropyl]thiophene-2-carboxamide

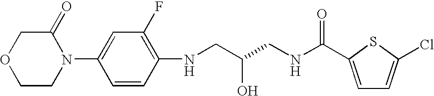

600 mg (2.69 mmol) of magnesium perchlorate were added to a solution of 376 mg (1.79 mmol) of the product from Example 7A and 429 mg (1.97 mmol) of the compound from Example 4A in 10 ml of acetonitrile, and the mixture was stirred at room temperature for 15 hours. Water was added, and the mixture was extracted with ethyl acetate. The organic extract was washed successively with water and saturated sodium chloride solution and dried over anhydrous magnesium sulfate. After filtration, the solvent was removed on a rotary evaporator. The residue was purified by preparative HPLC (Method 5). This gave 503 mg (64% of theory) of the title compound.

¹H-NMR (400 MHz, DMSO-d₆, δ/ppm): 8.61 (t, 1H), 7.68 (d, 1H), 7.18 (d, 1H), 7.11 (dd, 1H), 6.97 (dd, 1H), 6.73 (dd, 1H), 5.33 (t, 1H), 5.14 (d, 1H), 4.13 (s, 2H), 3.92 (dd, 2H), 3.87-3.79 (m, 1H), 3.63 (dd, 2H), 3.39-3.22 (m, 2H, partly superposed by the water signal), 3.21-3.15 (m, 1H), 3.08-3.02 (m, 1H).

HPLC (Method 1): Rₜ=3.75 min.

MS (DCl, NH₃, m/z): 428/430 (³⁵Cl/³⁷Cl) (M+H)⁺, 445/447 (M+NH₄)⁺.

### Example 9A

### 5-Chloro-N-({(5S)-3-[2-fluoro-4-(3-oxomorpholin-4-yl)phenyl]-2-oxo-1,3-oxazolidin-5-yl}methyl)thiophene-2-carboxamide

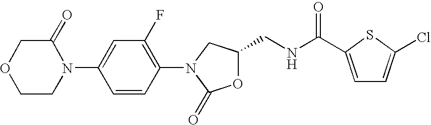

Method 1:

2.7 mg (0.022 mmol) of 4-dimethylaminopyridine were added to a solution of 478 mg (1.12 mmol) of the product from Example 8A and 363 mg (2.24 mmol) of carbonyldiimidazole in 10 ml of butyronitrile, and the mixture was heated at 70° C. After three days, the solvent was removed on a rotary evaporator. The product was isolated from the residue by preparative HPLC (Method 5). This gave 344 mg (68% of theory) of the title compound.

¹H-NMR (400 MHz, DMSO-d₆, δ/ppm): 8.98 (t, 1H), 7.70 (d, 1H), 7.52 (dd, 1H), 7.48 (dd, 1H), 7.31 (dd, 1H), 7.21 (d, 1H), 4.91-4.84 (m, 1H), 4.21 (s, 2H), 4.12 (t, 1H), 3.98 (dd, 2H), 3.80 (dd, 1H), 3.76 (dd, 2H), 3.68-3.57 (m, 2H).

HPLC (Method 1): Rₜ=3.82 min.

MS (DCl, NH₃, m/z): 471/473 (³⁵Cl/³⁷Cl) (M+NH₄)⁺.

Method 2:

At 0° C., 7.9 g (43 mmol, 1.2 eq.) of the compound from Example 1A were added to a solution of 11.2 g (36 mmol) of the compound from Example 62A in 224 ml of pyridine. After 30 min, the reaction mixture was concentrated under reduced pressure and the residue was taken up in water and dichloromethane. After phase separation, the aqueous phase was extracted twice with dichloromethane. The combined organic phases were washed with water and with saturated aqueous sodium chloride solution, dried over sodium sulfate, filtered and concentrated under reduced pressure. The residue was titrated with dichloromethane, filtered and dried under reduced pressure, which gave 7.4 g (45% of theory) of the title compound. The filtrate was concentrated under reduced pressure and the residue was purified by flash chromatography (silica gel 60, dichloromethane/methanol 100:1→100:2), which gave a further 1.9 g (12% of theory) of the title compound.

HPLC (Method 2): Rₜ=3.74 min;

MS (ESIpos): m/z =454 [M+H]⁺;

¹H-NMR (500 MHz, DMSO-d₆): δ=8.94 (t, 1H), 7.69 (d, 1H), 7.52 (dd, 1H), 7.48 (dd, 1H), 7.31 (dd, 1H), 7.20 (d, 1H), 4.92-4.84 (m, 1H), 4.21 (s, 2H), 4.12 (t, 1H), 3.97 (t, 2H), 3.81 (dd, 1H), 3.76 (t, 2H), 3.67-3.56 (m, 2H);

Melting points: 177° C., ΔH 84 Jg⁻¹ and 183° C., ΔH 7 Jg⁻¹.

### Example 10A

### 5-Chloro-N-(4-chlorobutanoyl)-N-({(5S)-2-oxo-3-[2-fluoro-4-(3-oxomorpholin-4-yl)phenyl]-1,3-oxazolidin-5-yl}methyl)thiophene-2-carboxamide

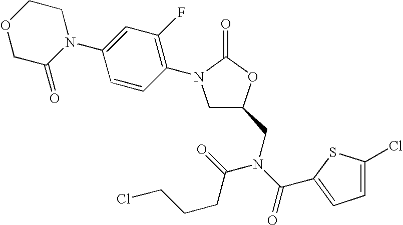

Under argon, 400 mg (0.88 mmol) 5-chloro-N-({(5S)-3-[2-fluoro-4-(3-oxomorpholin-4-yl)phenyl]-2-oxo-1,3-oxazolidin-5-yl}methylythiophene-2-carboxamide [compound (A)] were dissolved in 40 ml of absolute DMF. 1.677 g (1.76 mmol) of sodium hydride (98% pure) were added, and the mixture was stirred at RT for 20 min. 461 mg (11.9 mmol) of chlorobutanoyl chloride were then added, with the reaction temperature being maintained at RT. The mixture was stirred at RT for 16 h, and a little water was then added and the mixture was subsequently poured into saturated aqueous ammonium chloride solution/ethyl acetate 1:1. The phases were separated and the ethyl acetate phase was extracted first with sodium bicarbonate solution and then with saturated sodium chloride solution and subsequently dried over magnesium sulfate and concentrated. The residue was purified by preparative HPLC (Method 11). The diacylated compound obtained, which is formed after enolization, was stirred in 5 ml of a saturated solution of hydrogen chloride in dichloromethane overnight.

The mixture was then concentrated under reduced pressure, and the residue was dried under high vacuum. This gave 20 mg (4% of theory) of the target compound.

HPLC (Method 10): Rₜ=1.8 min;

LC-MS (Method 7): Rₜ=1.2 min; m/z=558 (M+H)⁺.

### Example 11A

### 5-Chloro-N-(4-chloropentanoyl)-N-({(5S)-2-oxo-3-[2-fluoro-4-(3-oxomorpholin-4-yl)phenyl]-1,3-oxazolidin-5-yl}methyl)thiophene-2-carboxamide

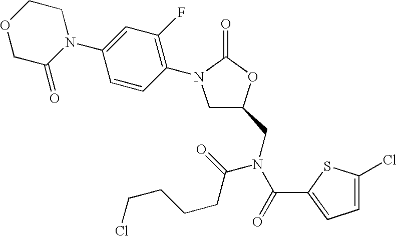

Under argon, 100 mg (0.22 mmol) of 5-chloro-N-({(5S)-3-[2-fluoro-4-(3-oxomorpholin-4-yl)phenyl]-2-oxo-1,3-oxazolidin-5-yl}methylythiophene-2-carboxamide [compound (A)] were dissolved in 10 ml of absolute DMF. 11 mg (0.44 mmol) of sodium hydride (98% pure) were added, and the mixture was stirred at RT for 20 min. 461 mg (2.98 mmol) of chloropentanoyl chloride were then added, with the reaction temperature being maintained at RT. The mixture was stirred at RT for 16 h and then poured into saturated aqueous ammonium chloride solution/ethyl acetate 1:1. The phases were separated and the ethyl acetate phase was extracted first with sodium bicarbonate solution and then with saturated sodium chloride solution and then dried over magnesium sulfate and concentrated. The residue was purified by preparative HPLC (Method 11). The diacylated compound obtained, which is formed after enolization, was stirred in 2 ml of a saturated solution of hydrogen chloride and dichloromethane overnight. The mixture was then concentrated under reduced pressure and the residue was dried under high vacuum. This gave 20 mg (17% of theory) of the target compound.

HPLC (Method 10): Rₜ=1.9 min;

LC-MS (Method 6): Rₜ=2.4 min; m/z=572 (M+H)⁺.

### Example 12A

### N-(4-Aminobutanoyl)-5-chloro-N-({(5S)-2-oxo-3-[2-fluoro-4-(3-oxomorpholin-4-yl)phenyl]-1,3-oxazolidin-5-yl}methyl)thiophene-2-carboxamide hydrochloride

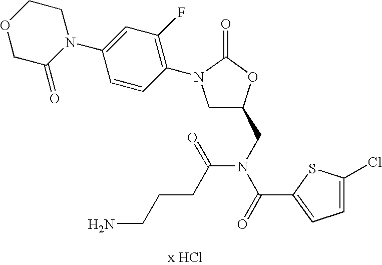

Step a):

Under an atmosphere of argon, 0.5 g (1.1 mmol) of the compound (A) was dissolved in 27 ml of DMF, 79 mg (3.31 mmol) of sodium hydride was added and the mixture was stirred at RT for 30 min. 4.14 g (11 mmol) of the freshly prepared compound from Example 14A, dissolved in 3 ml of DMF, were then added. The mixture was stirred at RT for a further 15 min, and 1 ml of methanol was then added. The mixture was poured into a 1:1 mixture of 10% strength sodium bicarbonate solution and ethyl acetate. The organic phase was separated off and washed two more times with 10% strength sodium bicarbonate solution. The organic phase was then concentrated, and the residue was, at RT, stirred with 5 ml of a saturated solution of hydrogen chloride in dichloromethane for 20 h, resulting in the enol ester initially formed being cleaved. The mixture was then concentrated, and the residue that remained was purified by flash chromatography on silica gel using the mobile phase toluene/ethyl acetate, the mixing ratio being increased from 1:1 via 1:2 to 1:3. The appropriate fractions were concentrated, giving 124 mg (8% of theory) of the doubly protected intermediate as a foam.

HPLC (Method 10): Rₜ=2.3 min;

LC-MS (Method 8): Rₜ=2.33 min; m/z=793 (M+H)⁺.

Step b):

At RT, 118 mg (0.149 mmol) of the intermediate obtained above were stirred in 6 ml of anhydrous trifluoroacetic acid overnight. The mixture was then concentrated under high vacuum, with the temperature being maintained at about 20° C. The residue was taken up in 50 ml of aqueous hydrochloric acid which had been adjusted to pH 3, and 75 ml of dichloromethane were added to the solution. The mixture was mixed by shaking and the aqueous phase was then separated off and concentrated under high vacuum. The residue was purified by preparative HPLC (Method 11). The appropriate fractions were combined, concentrated and then lyophilized from 1N hydrochloric acid. Yield: 59 mg (69% of theory)

HPLC (Method 10): Rₜ=0.98 min;

LC-MS (Method 8): Rₜ=0.98 min; m/z=539 (M+H)⁺.

### Example 13A

### N-(5-Aminopentanoyl)-5-chloro-N-({(5S)-2-oxo-3-[2-fluoro-4-(3-oxomorpholin-4-yl)phenyl]-1,3-oxazolidin-5-yl}methyl)thiophene-2-carboxamide hydrochloride

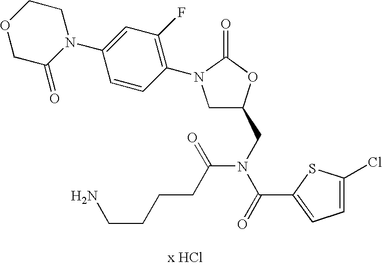

The title compound can be prepared analogously to Example 12A from compound (A) and the compound from Example 19A.

### Example 14A

### Benzyl (4-chloro-4-oxobutyl)(4-methoxybenzyl)carbamate

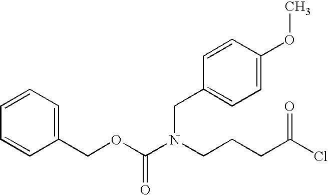

The preparation was carried out analogously to Example 19A starting with 4-aminobutyric acid.

### Example 15A

### (2S)-2-[(tert-Butoxycarbonyl)amino]-3-methylbutanethioic S-acid

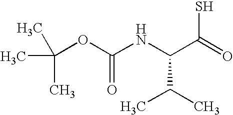

The title compound was prepared from Boc-valine analogously to a procedure known from the literature [R. Michelot et al., Bioorg. Med. Chem. 1996, 4, 2201].

### Example 16A

### [(tert-Butoxycarbonyl)amino]ethanethioic S-acid

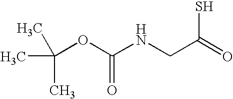

The title compound was prepared from Boc-glycine analogously to a procedure known from the literature [R. Michelot et al., Bioorg. Med. Chem. 1996, 4, 2201].

### Example 17A

### (2S)-2,6-Bis[(tert-butoxycarbonyl)amino]hexanethio S-acid

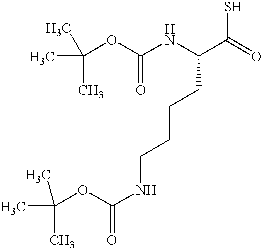

The title compound was prepared from Bis-Boc-lysine analogously to a procedure known from the literature [R. Michelot et al., Bioorg. Med. Chem. 1996, 4, 2201].

### Example 18A

### (2S )-2-[(tert-Butoxycarbonyl)amino]propanethio S-acid

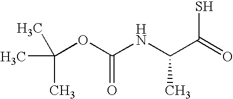

The title compound was prepared from Boc-alanine analogously to a procedure known from the literature [R. Michelot et al., Bioorg. Med. Chem. 1996, 4, 2201].

### Example 19A

### Benzyl (5-chloro-5-oxopentyl)(4-methoxybenzyl)carbamate

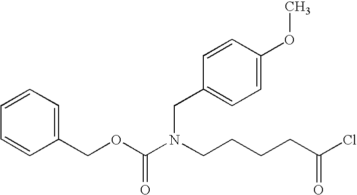

10 g (85.4 mmol) of 5-aminovaleric acid, 17.4 g (128 mmol) of p-anisaldehyde and 10.3 g (85.4 mmol) of magnesium sulfate were taken up in 330 ml of ethanol and heated under reflux for 1 h. The mixture was filtered off, the filter residue was washed with ethanol and 1.94 g (51.2 mmol) of sodium borohydride was then added a little at a time over a period of 15 min to the solution. Initially, 10 ml of water were added, and then 128 ml of a 2 M aqueous sodium hydroxide solution. After 5 min, the mixture was diluted with 300 ml of water and then extracted three times with in each case 200 ml of ethyl acetate. The aqueous phase was adjusted to pH 2 using 4 M hydrochloric acid and concentrated under reduced pressure. The residue was purified by flash chromatography on silica gel using the mobile phase acetonitrile/water/acetic acid 5:1:0.1. The appropriate fractions were concentrated and triturated with ethyl acetate and diethyl ether. The residue was then filtered off with suction and dried under high vacuum. This gave 9.1 g (45% of theory) of the p-methoxybenzyl-protected amino acid.

The amino acid was taken up in 1.6 l dioxane/water 1:1 and adjusted to pH 10 using aqueous sodium hydroxide solution, and 12.97 g (76 mmol) of benzyl chlorocarbonate were then added dropwise. After 15 min of stirring at RT, the dioxane was removed under reduced pressure and the solution that remained was adjusted to pH 2 using 2 M hydrochloric acid. The mixture was extracted with ethyl acetate and the organic phase was then washed twice with water. The organic phase was then concentrated and the residue was dried under high vacuum. This was followed by purification by flash chromatography on silica gel using the mobile phase acetonitrile. The appropriate fractions were concentrated and the residue was dried under high vacuum. This gave 5.6 g (38% of theory) of the protected amino acid.

LC-MS (Method 6): Rₜ=2.47 min; m/z=372 (M+H)⁺.

5.6 g (15 mmol) of 5-{[(benzyloxy)carbonyl](4-methoxybenzyl)amino}valeric acid were dissolved in 60 ml of dichloromethane and 2.2 ml of thionyl chloride were added. The mixture was heated under reflux for 30 min. The mixture was then concentrated under reduced pressure, more dichloromethane was added to the residue and the mixture was concentrated again. What remained was a viscous oil which was dried under high vacuum. This gave 5.7 g (98% of theory) of the target compound which was further reacted without further purification and characterization.

### Exemplary Embodiments

General Procedure 1 for preparing cesium salts of carboxylic acids or suitably protected amino acid derivatives:

1 mmol of the appropriate carboxylic acid is dissolved in a mixture of 10 ml of dioxane and 10 ml of water, and 0.5 mmol of cesium carbonate is added. This is followed by lyophilization.

General procedure 2 for preparing urethane-protected N-carboxy anhydrides of suitably protected amino acid derivatives:

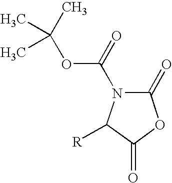

Urethane-protected N-carboxy anhydrides of amino acid derivatives are either commercially available or can be prepared according to literature procedures: M. Johnston et al. J. Org. Chem. 1985, 50, 2200; W. D. Fuller et al. J. Am. Chem. Soc. 1990, 112, 7414; S. Mobasheri et al. J. Org. Chem. 1992, 57, 2755.

General procedure 3 for preparing N-hydroxysuccinimide esters of suitably protected amino acid derivatives:

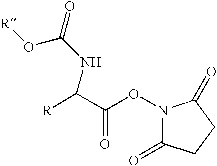

N-Hydroxysuccinimide esters of amino acid derivatives are either commercially available or can be prepared by standard methods of peptide chemistry.

### Example 1

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-4-oxobutyl glycinate hydrochloride

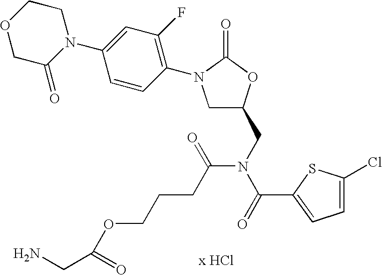

The title compound can be prepared analogously to Examples 2 and 25 from the compound from Example 10A and Boc-glycine.

### Example 2

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-5-oxopentyl glycinate hydrochloride

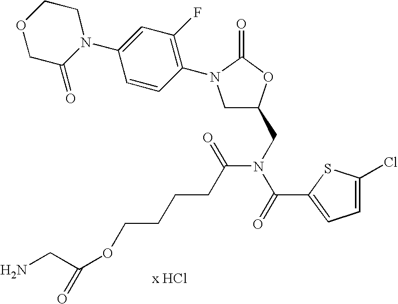

The title compound was prepared by dissolving 5 mg of the compound from Example 25 in aqueous hydrochloric acid adjusted to pH 3, followed by lyophilization. Yield: 4.4 mg (99% of theory)

HPLC (Method 10): Rₜ=1.3 min;

LC-MS (Method 8): Rₜ=1.08 min; m/z=611 (M+H)⁺.

### Example 3

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-5-oxopentyl L-valinate hydrochloride

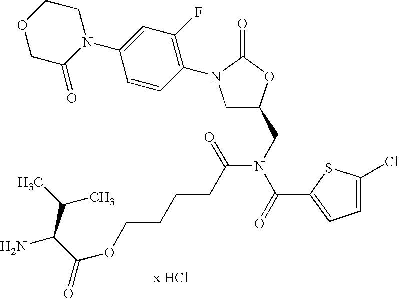

The title compound can be prepared analogously to Examples 2 and 25 from the compound from Example 11A and Boc-valine.

### Example 4

### S-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl) (2S)-2-amino-3-methylbutanethioate hydrochloride

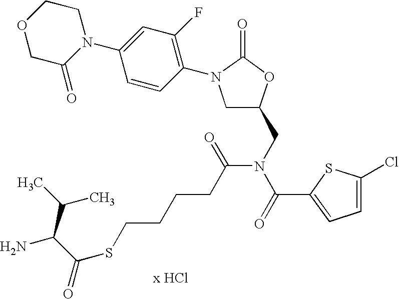

The title compound was prepared by dissolving 7.4 mg of the compound from Example 26 in aqueous hydrochloric acid adjusted to pH 3, followed by lyophilization. Yield: 6.4 mg (quant.)

HPLC (Method 10): Rₜ=1.6 min;

LC-MS (Method 6): Rₜ=1.52 min; m/z=669 (M+H)⁺.

### Example 5

### S-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl) aminoethanethioate hydrochloride

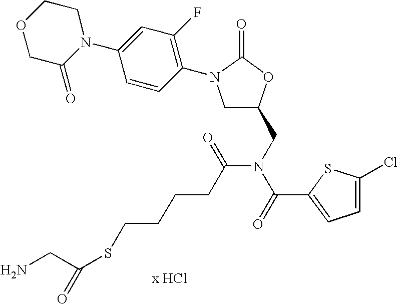

The title compound can be prepared analogously to Examples 4 and 26 from the compounds of Examples 11A and 16A.

### Example 6

### S-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl) (2S)-2,6-diaminohexanethioate dihydrochloride

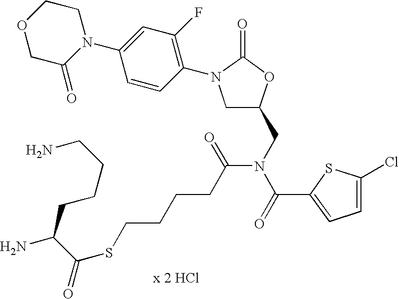

The title compound can be prepared analogously to Examples 4 and 26 from the compounds of Examples 11A and 17A.

### Example 7

### S-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl) (2S)-2-aminopropanethioate hydrochloride

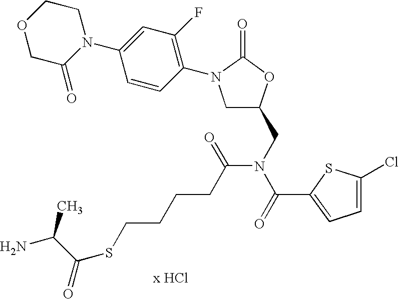

The title compound can be prepared analogously to Examples 4 and 26 from the compounds of Examples 11A and 18A.

### Example 8

### S-(5-{[(5-Chloro-2-thienyl)carbonyl]({(5S)-2-oxo-3-[2-fluoro-4-(3-oxomorpholiin-4-yl)phenyl]-1,3-oxazolidin-5-yl}methyl)amino}-4-oxobutyl) (2S)-2-amino-3-methylbutanethioate hydrochloride

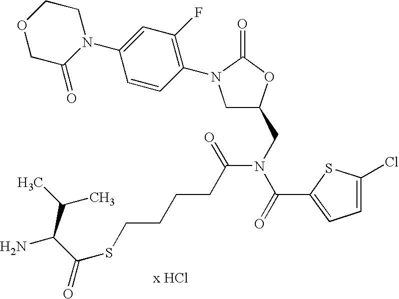

The title compound can be prepared analogously to Examples 4 and 26 from the compounds of Examples 10A and 15A.

### Example 9

### 5-Chloro-N-[4-(glycylamino)butanoyl]-N-({(5S)-2-oxo-3-[2-fluoro-4-(3-oxomorpholin-4-yl)phenyl]-1,3-oxazolidin-5-yl}methyl)thiophene-2-carboxamide hydrochloride

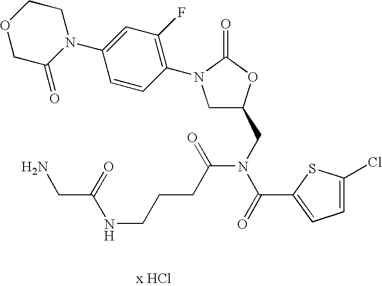

The title compound can be prepared analogously to Example 23 from the compound from Example 12A using Boc-glycine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 10

### 5-Chloro-N-[4-(glycylamino)pentanoyl]-N-({(5S)-2-oxo-3-[2-fluoro-4-(3-oxomorpholin-4-yl)phenyl]-1,3-oxazolidin-5-yl}methyl)thiophene-2-carboxamide hydrochloride

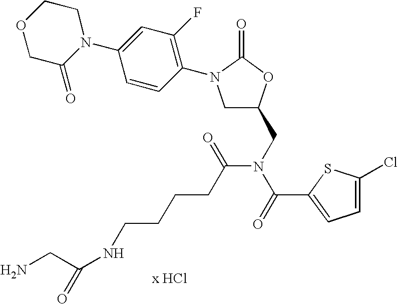

The title compound can be prepared analogously to Example 23 from the compound from Example 13A using Boc-glycine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 11

### N-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl)-L-prolinamide hydrochloride

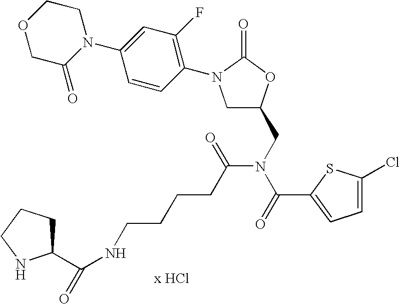

The title compound can be prepared analogously to Example 23 from the compound from Example 13A using Boc-proline. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 12

### N-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl)-L-histidinamide hydrochloride

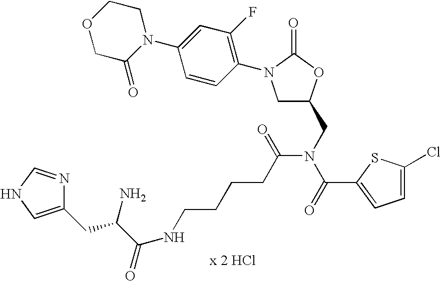

The title compound can be prepared analogously to Example 23 from the compound from Example 13A using bis-Boc-histidine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 13

### N-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl)-L-valinamide hydrochloride

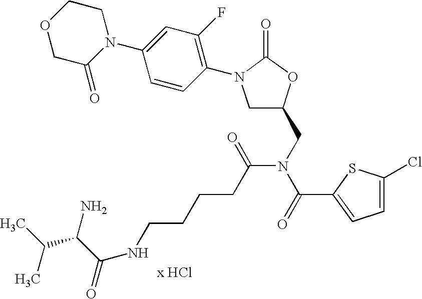

The title compound can be prepared analogously to Example 23 from the compound from Example 13A using Boc-valine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 14

### N-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl)-L-lysinamide hydrochloride

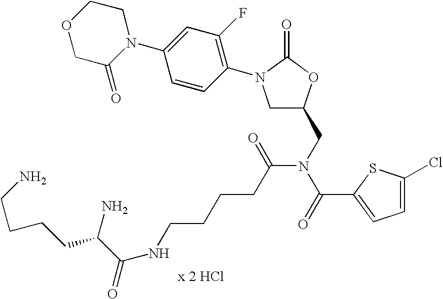

The title compound can be prepared analogously to Example 23 from the compound from Example 13A using bis-Boc-lysine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 15

### 5-Chloro-N-({(5S)-2-oxo-3-[2-fluoro-4-(3-oxomorpholin-4-yl)phenyl]-1,3-oxazolidin-5-yl}methyl)-N-[5-(L-threonylamino)pentanoyl]thiophene-2-carboxamide hydrochloride

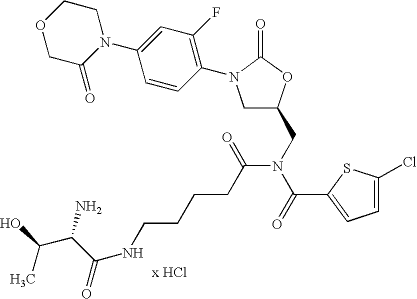

The title compound can be prepared analogously to Example 23 from the compound from Example 13A using Boc-threonine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 16

### N-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl)-L-tyrosinamide hydrochloride

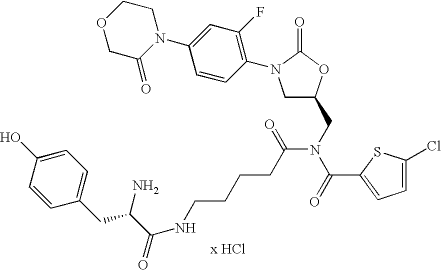

The title compound can be prepared analogously to Example 23 from the compound from Example 13A using Boc-tyrosine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 17

### N¹-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl)-L-aspartamide hydrochloride

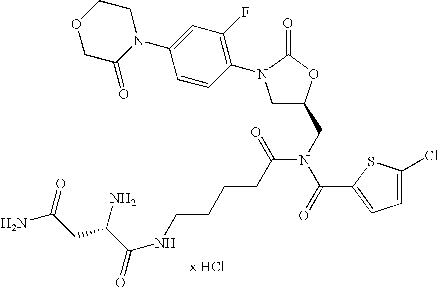

The title compound can be prepared analogously to Example 23 from the compound from Example 13A using Boc-asparagine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 18

### N-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl)-L-phenylalaninamide hydrochloride

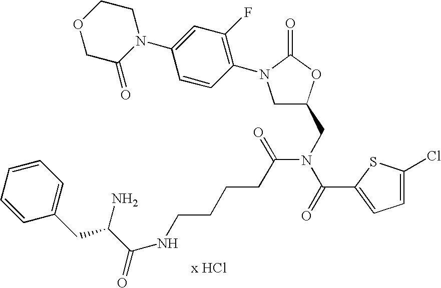

The title compound can be prepared analogously to Example 23 from the compound from Example 13A using Boc-phenylalanine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 19

### N¹-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl)-L-glutamamide hydrochloride

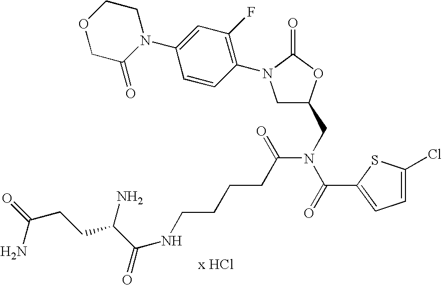

The title compound can be prepared analogously to Example 23 from the compound from Example 13A using Boc-glutamine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 20

### N-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl)-L-alpha-glutamine hydrochloride

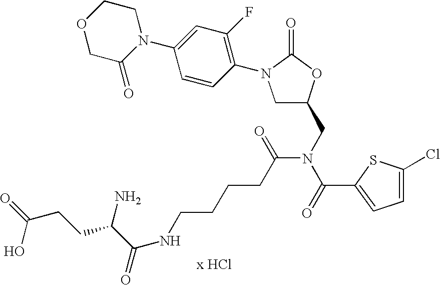

The title compound can be prepared analogously to Example 23 from the compound from Example 13A using tert-butyl Boc-glutamate. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 21

### 5-Chloro-N-({(5S)-2-oxo-3-[2-fluoro-4-(3-oxomorpholin-4-yl)phenyl]-1,3-oxazolidin-5-yl}methyl)-N-[5-(L-serylamino)pentanoyl]thiophene-2-carboxamide hydrochloride

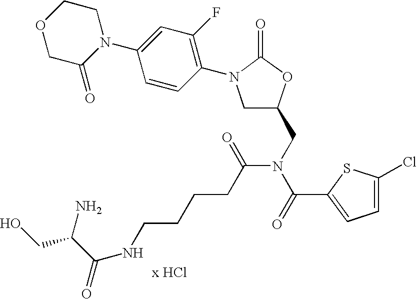

The title compound can be prepared analogously to Example 23 from the compound from Example 13A using Boc-serine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 22

### N-(5-{[(5-Chloro-2-thienyl)carbonyl]({(5S)-2-oxo-3-[2-fluoro-4-(3-oxomorpholiin-4-yl)phenyl]-1,3-oxazolidin-5-yl}methyl)amino}-5-oxopentyl)-L-leucinamide hydrochloride

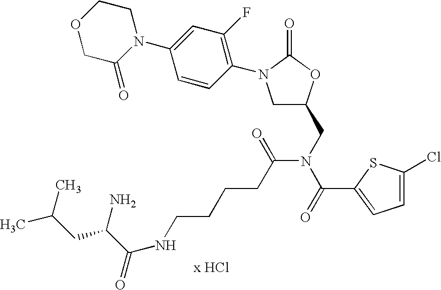

The title compound can be prepared analogously to Example 23 from the compound from Example 13A using Boc-leucine. Alternatively, the deprotection can also be carried out as described in Example 25, Step b) using trifluoroacetic acid, and the title compound can be generated from the trifluoroacetate initially formed by double decomposition with aqueous hydrochloric acid.

### Example 23

### N-(5-{[(5-Chloro-2-thienyl)carbonyl]({(5S)-2-oxo-3-[2-fluoro-4-(3-oxomorpholiin-4-yl)phenyl]-1,3-oxazolidin-5-yl}methyl)amino}-5-oxobutyl)-L-histidinamide hydrochloride

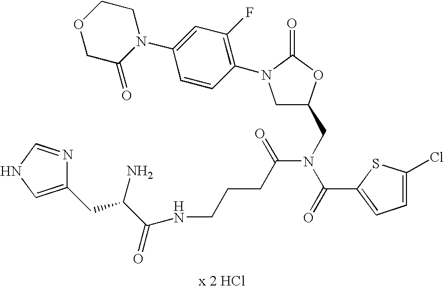

Step a):

59 mg (0.103 mmol) of the compound from Example 12A were initially charged in 15 ml of DMF. 51 mg (0.144 mmol) of N,1-bis(tert-butoxycarbonyl)-L-histidine, 19 mg (0.123 mmol) of 1-hydroxy-1H-benzotriazole and 24 mg (0.123 mmol) of N-(3-dimethylaminopropyl)-N′-ethylcarbodiimide hydrochloride and also 12 mg of ethyldiisopropylamine were then added, and the mixture was stirred at RT overnight. The mixture was then poured into a 1:1 mixture of saturated ammonium chloride solution and ethyl acetate. The organic phase was separated off and washed with 10% strength sodium bicarbonate solution and saturated aqueous sodium chloride solution and then dried over magnesium sulfate. The organic phase was then concentrated, and the residue was dried under high vacuum. This gave 66 mg (55% of theory) of a foam of the mono-Boc-protected intermediate.

HPLC (Method 10): Rₜ=1.2 min

LC-MS (Method 13): Rₜ=0.93 min; m/z=776 (M+H)⁺

Step b):

66 mg (0.085 mmol) of the mono-Boc-protected intermediate were dissolved in 3 ml of a saturated solution of hydrogen chloride in dichloromethane and shaken at RT for 30 min. The resulting precipitate was filtered off with suction and extracted with the mother liquor. The aqueous phase was concentrated and purified by preparative HPLC (Method 11). The appropriate fractions were combined and concentrated, and the residue was lyophilized from 1N hydrochloric acid. This gave 3 mg (4% of theory) of the title compound.

HPLC (Method 10): Rₜ=1.07 min;

LC-MS (Method 8): Rₜ=1 min; m/z=676 (M+H)⁺.

### Example 24

### N-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl)-L-alpha-asparagine hydrochloride

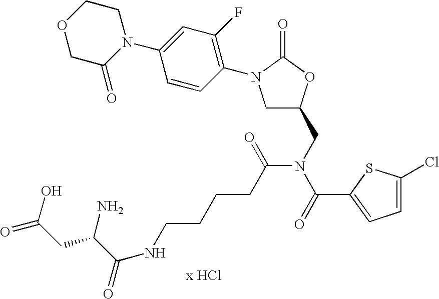

The title compound can be prepared analogously to Example 23 from the appropriate starting materials.

### Example 25

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-5-oxopentyl glycinate trifluoroacetate

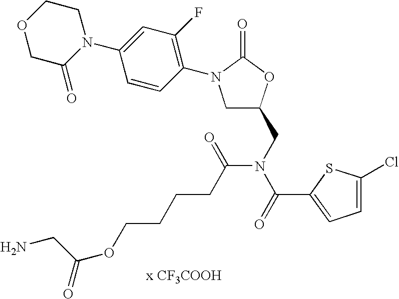

Step a):

48 mg (0.084 mmol) of the compound from Example 11A and 77 mg (0.252 mmol) of the cesium salt of Boc-glycine (prepared from Boc-glycine according to the General Procedure 1) were dissolved in 10 ml of DMF. After 3 h of stirring at 60° C., once more the same amount of cesium salt was added and the mixture was stirred at 60° C. overnight. The mixture was then poured into a 1:1 mixture of saturated aqueous ammonium chloride solution and ethyl acetate. The organic phase was separated off and washed with 10% strength sodium bicarbonate solution and saturated aqueous sodium chloride solution and then dried over magnesium sulfate. The organic phase was then concentrated and the residue was purified by flash chromatography on silica gel using, as mobile phase, initially dichloromethane/ethyl acetate 3:1 and then dichloromethane/ethyl acetate/methanol 15:5:1. The appropriate fractions were combined, the solvent was evaporated and the residue was then dried under high vacuum. This gave 10 mg (16% of theory) of the Boc-protected intermediate.

HPLC (Method 10): Rₜ=1.9 min;

LC-MS (Method 8): Rₜ=1.98 min; m/z=711 (M+H)⁺.

Step b):

9 mg (0.013 mmol) of the protected compound were dissolved in 1.5 ml of dichloromethane, 1.5 ml of anhydrous trifluoroacetic acid were added and the mixture was then stirred at RT for 15 min. The mixture was then concentrated under reduced pressure, and the residue was lyophilized from dioxane/water. This gave 8 mg (85% of theory) of the title compound.

HPLC (Method 10): Rₜ=1.3 min;

LC-MS (Method 13): Rₜ=0.83 min; m/z=611 (M+H)⁺.

### Example 26

### S-(5-{[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino}-5-oxopentyl) (2S)-2-amino-3-methylbutanethioate trifluoroacetate

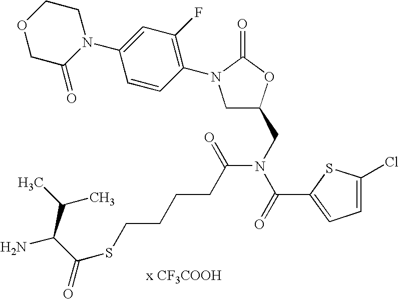

Step a):

47 mg (0.082 mmol) of the compound from Example 11A and 90 mg (0.25 mmol) of the cesium salt of (2S)-2-[(tert-butoxycarbonyl)amino]-3-methylbutanethio S-acid (prepared from Example 15A according to the General Procedure 1) were dissolved in 2 ml of DMF. After 3 h of stirring at 60° C., once more the same amount of cesium salt was added and the mixture was stirred at 60° C. overnight. The mixture was poured into a 1:1 mixture of saturated aqueous ammonium chloride solution and ethyl acetate. The organic phase was separated off and washed with 10% strength sodium bicarbonate solution and saturated aqueous sodium chloride solution and then dried over magnesium sulfate. The organic phase was then concentrated and the residue was purified by flash chromatography on silica gel, using, as mobile phase, initially dichloromethane/ethyl acetate 3:1 and then dichloromethane/ethyl acetate/methanol 15:5:1. The appropriate fractions were combined, and the solvent was evaporated. This purification process was repeated a second time. The residue that remained was then purified again by preparative HPLC (Method 11). The appropriate fractions were combined and the solvent was evaporated. This gave 7 mg (11% of theory) of the Boc-protected intermediate.

HPLC (Method 10): Rₜ=2.3 min;

LC-MS (Method 13): Rₜ=1.43 min; m/z=769 (M+H)⁺.

Step b):

7 mg (0.009 mmol) of the protected compound were dissolved in 1 ml of dichloromethane, 1 ml of anhydrous trifluoroacetic acid was added and the mixture was then stirred at RT for 15 min. The mixture was then concentrated under reduced pressure and the residue was lyophilized from acetonitrile/water. This gave 6.8 mg (95% of theory) of the title compound.

HPLC (Method 10): Rₜ=1.6 min;

LC-MS (Method 8): Rₜ=1.24 min; m/z=669 (M+H)⁺.

### B. DETERMINATION OF SOLUBILITY, STABILITY AND LIBERATION BEHAVIOR

a) Determination of the Solubility:

The test substance is suspended in water or dilute hydrochloric acid (pH 4). This suspension is shaken at room temperature for 24 h. After ultracentrifugation at 224 000 g for 30 min, the supernatant is diluted with DMSO and analyzed by HPLC. A two-point calibration plot of the test compound in DMSO is used for quantification.

HPLC Method:

Agilent 1100 with DAD (G1315A), quat. pump (G1311A), autosampler CTC HTS PAL, degasser (G1322A) and column thermostat (G1316A); column: Zorbax Extend-C18 3.5μ; temperature: 40° C.; mobile phase A: water+5 ml of perchloric acid/liter, mobile phase B: acetonitrile; flow rate: 0.7 ml/min; gradient: 0-0.5 min 98% A, 2% B; ramp 0.5-4.5 min 10% A, 90% B; 4.5-6 min 10% A, 90% B; ramp 6.5-6.7 min 98% A, 2% B; 6.7-7.5 min 98% A, 2% B.

b) Stability in Buffer at Various pH Values:

0.25 mg of the test substance is weighed into a 2 ml HPLC vial and 0.5 ml of acetonitrile is added. The substance is dissolved by putting the sample vessel in an ultrasonic bath for about 10 seconds. Then 0.5 ml of the respective buffer solution is added, and the sample is again treated in the ultrasonic bath.

Buffer Solutions Employed:

pH 4.0: 1 liter of Millipore water is adjusted to pH 4.0 with 1 N hydrochloric acid;

pH 7.4: 90 g of sodium chloride, 13.61 g of potassium dihydrogen phosphate and 83.35 g of 1 M sodium hydroxide solution are made up to 1 liter with Millipore water and then diluted 1:10.

10 μl portions of the test solution are analyzed by HPLC for their content of unchanged test substance every hour over a period of 24 hours at 37° C. The percentage areas of the appropriate peaks are used for quantification.

HPLC Method:

Agilent 1100 with DAD (G1314A), binary pump (G1312A), autosampler (G1329A), column oven (G1316A), thermostat (G1330A); column: Kromasil 100 C18, 125 mm×4 mm, 5 μm; column temperature: 30° C.; mobile phase A: water+5 ml of perchloric acid/liter, mobile phase B: acetonitrile.

Gradient:

0-1.0 min 98% A, 2% B→1.0-13.0 min 50% A, 50% B→13.0-17.0 min 10% 90% B→17.0-18.0 min 10% A, 90% B→18.0-19.5 98% A, 2% B→19.5-23.0 min 98% A, 2% B; flow rate: 2.0 ml/min; UV detection: 210 nm.

c) In vitro Stability in Rat Plasma and Human Llasma (HPLC Detection):

0.5 mg of substance is dissolved in 1 ml of dimethyl sulfoxide/water 1:1. 500 μl of this sample solution are mixed with 500 μl of rat plasma at 37° C. and shaken. A first sample (10 μl) is immediately taken for HPLC analysis. In the period up to 2 h after the start of incubation, further aliquots are taken after 2, 5, 10, 30, 60 and 90 min, and the contents of the respective test substance and of the active ingredient compound (A) liberated therefrom are determined.

HPLC-Method:

Agilent 1100 with DAD (G1314A), binary pump (G1312A), autosampler (G1329A), column oven (G1316A), thermostat (G1330A); column: Kromasil 100 C18, 250 mm×4.6 mm, 5 μm; column temperature: 30° C.; mobile phase A: water +5 ml of perchloric acid/liter, mobile phase B: acetonitrile.

Gradient:

0-3.0 min 69% A, 31% B→3.0-18.0 min 69% A, 31% B→18.0-20.0 min 10% A, 90% B→20.0-21.0 90% A, 10% B→21.0-22.5.0 min 98% A, 2% B→22.5-25.0 min 98% A, 2% B; flow rate: 2.0 ml/min; UV detection: 248 nm.

d) In vitro Stability in Rat and Human Plasma (LC/MS-MS Detection):

A defined plasma volume (e.g. 2.0 ml) is warmed to 37° C. in a closed test tube in a waterbath. After the intended temperature is reached, a defined amount of the test substance is added as solution (volume of the solvent not more than 2% of the plasma volume). The plasma is shaken and a first sample (50-100 μl) is immediately taken. Then 4-6 further aliquots are taken in the period up for 2 h after the start of incubation.

Acetonitrile is added to the plasma samples to precipitate proteins. After centrifugation, the test substance and, where appropriate, known cleavage products of the test substance in the supernatant are determined quantitatively with a suitable LC/MS-MS method.

Determinations of stability in heparinized rat or human blood are carried out as described for plasma.

e) i.v. Pharmacokinetics in Wistar Rats:

On the day before administration of the substance, a catheter for obtaining blood is implanted in the jugular vein of the experimental animals (male Wistar rats, body weight 200-250 g) under Isofluran® anesthesia.

On the day of the experiment, a defined dose of the test substance is administered as solution into the tail vein using a Hamilton® glass syringe (bolus administration, duration of administration <10 s). Blood samples (8-12 time points) are taken through the catheter sequentially over the course of 24 h after administration of the substance. Plasma is obtained by centrifuging the samples in heparinized tubes. Acetonitrile is added to a defined plasma volume per time point to precipitate proteins. After centrifugation, test substance and, where appropriate, known cleavage products of the test substance in the supernatant are determined quantitatively using a suitable LC/MS-MS method.

The measured plasma concentrations are used to calculate pharmacokinetic parameters of the test substance and of the active ingredient compound (A) liberated therefrom, such as AUC, Cₘₐₓ, T_{1/2} (half-life) and CL (clearance).

f) Hepatocyte Assay to Determine the Metabolic Stability:

The metabolic stability of the test compounds in the presence of hepatocytes is determined by incubating the compounds at low concentrations (preferably below 1 μM) and with low cell counts (preferably with 1×10⁶ cells/ml) in order to ensure as far as possible linear kinetic conditions in the experiment. Seven samples of the incubation solution are taken in a fixed time pattern for the LC-MS analysis in order to determine the half-life (i.e. the degradation) of the compound. Various clearance parameters (CL) and Fₘₐₓ values are calculated from this half-life (see below).

The CL and Fₘₐₓ values represent a measure of the phase 1 and phase 2 metabolism of the compound in the hepatocytes. In order to minimize the influence of the organic solvent on the enzymes in the incubation mixtures, its concentration is generally limited to 1% (acetonitrile) or 0.1% (DMSO).

A cell count for hepatocytes in the liver of 1.1×10⁸ cells/g of liver is used for calculation for all species and breeds. CL parameters calculated on the basis of half-lives extending beyond the incubation time (normally 90 minutes) can be regarded only as rough guidelines.

The calculated parameters and their meaning are:
Fₘₐₓ well-stirred [%] maximum possible bioavailability after oral administration
Calculation: (1-CL_{blood} well-stirred/QH)*100
CL_{blood} well-stirred [L/(h*kg)] calculated blood clearance (well stirred model)
Calculation: (QH*CL′_{intrinsic})/(QH+CL′_{intrinsic})
CL′_{intrinsic} [ml/(min*kg)] maximum ability of the liver (of the hepatocytes) to metabolize a compound (on the assumption that the hepatic blood flow is not rate-limiting)
Calculation: CL′_{intrinsic, apparent} * species-specific hepatocyte count [1.1*10⁸/g of liver]*species-specific liver weight [g/kg]
CL′_{intrinsic, apparent} [ml/(min*mg)] normalizes the elimination constant by dividing it by the cell count of hepatocytes employed x (x*10⁶/ml)
Calculation: kₑₗ[1/min]/(cell count [x*10⁶]/incubation volume [ml])
(QH=species-specific hepatic blood flow).

g) Determination of the Antithrombotic Effect in an Arteriovenous Shunt Model in Rats:

Fasting male rats (strain: HSD CPB:WU) are anesthetized by intraperitoneal administration of a Rompun/Ketavet solution (12 mg/kg/50 mg/kg). Thrombus formation is induced in an arteriovenous shunt based on the method described by P. C. Wong et al. [Thrombosis Research 83 (2), 117-126 (1996)]. For this purpose, the left jugular vein and the right carotid artery are exposed. An 8 cm-long polyethylene catheter (PE60, from Becton-Dickinson) is secured in the artery, followed by a 6 cm-long Tygon tube (R-3606, ID 3.2 mm, from Kronlab) which contains a roughened nylon thread (60×0.26 mm, from Berkley Trilene) made into a double loop to produce a thrombogenic surface. A 2 cm-long polyethylene catheter (PE60, from Becton-Dickinson) is secured in the jugular vein and connected by a 6 cm-long polyethylene catheter (PE160, from Becton-Dickinson) to the Tygon tube. The tubes are filled with physiological saline before the shunt is opened. The extracorporeal circulation is maintained for 15 min. The shunt is then removed and the nylon thread with the thrombus is immediately weighed. The empty weight of the nylon thread has been determined before the start of the experiment. The test substance (as solution in physiological saline adjusted to pH 4 with 0.1 N hydrochloric acid) is administered as bolus injection before attaching the extracorporeal circulation.

C. EXEMPLARY EMBODIMENTS OF PHARMACEUTICAL COMPOSITIONS

The compounds according to the invention can be converted for example into pharmaceutical preparations in the following way:

i.v. Solution:

The compound according to the invention is dissolved at a concentration below the saturation solubility in a physiologically tolerated solvent (e.g. isotonic saline, 5% glucose solution and/or 30% PEG 400 solution, each of which is adjusted to a pH of 3-5). The solution is sterilized by filtration where appropriate and/or dispensed into sterile and pyrogen-free injection containers.

1. A compound of the formula (I) 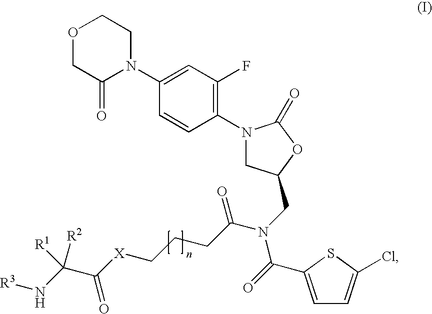
in which
n is the number 1 or 2,
X is an oxygen atom, sulfur atom or NH,
R¹ is the side group of a natural α-amino acid or its homologs or isomers,
R² is hydrogen or methyl,
R³ is hydrogen,
or
R¹ and R³ are linked via a (CH₂)₃ or (CH₂)₄ group and together with the nitrogen or carbon atom to which they are attached form a 5- or 6-membered ring, and the salts thereof.

2. The compound of the formula (I) as claimed in claim 1, in which n is the number 1 or 2,
X is an oxygen atom,sulfur atom or NH,
R¹ is hydrogen, methyl, propan-2-yl, propan-1-yl, 2-methylpropan-l-yl, imidazol-4-ylmethyl, hydroxymethyl, 1-hydroxyethyl, carboxymethyl, 2-carboxyethyl, carbamoylmethyl, 2-carbamoylethyl, 4-aminobutan-1-yl, 3-aminopropan-1-yl, 3-guanidinopropan-1-yl, benzyl or 4-hydroxybenzyl,
R² is hydrogen or methyl,
R³ is hydrogen,
or
R¹ and R³ are linked via a (CH₂)₃ or (CH₂)₄ group and together with the nitrogen or carbon atom to which they are attached from a 5- or 6-membered ring.

3. The compound of the formula (I) as claimed in claim 1, in which
n is the number 1 or 2,
X is NH,
R¹ is hydrogen, methyl, propan-2-yl, 2-methylpropan-1-yl, imidazol-4-ylmethyl, hydroxymethyl, 1-hydroxyethyl, carboxymethyl, 2-carboxyethyl, carbamoylmethyl, 2-carbamoylethyl, 4-aminobutan-1-yl, benzyl or 4-hydroxybenzyl,
R² is hydrogen,
R³ is hydrogen.

4. A process for preparing a compound of the formula (I) or one of the salts thereof according to claim 1, characterized in that [A] the compound of the formula 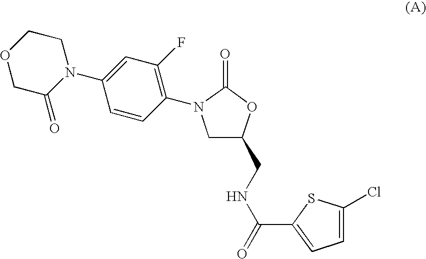
is initially converted in an inert solvent in the presence of a base with a compound of the formula 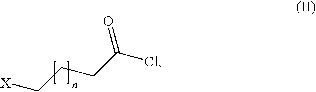
in which n has the meaning indicated in claim 1,
and
Q is a leaving group such as, for example, chlorine, bromine or iodine, into a compound of the formula 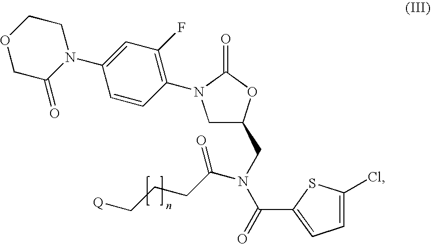
in which n has the meaning indicated in claim 1,
Q has the meaning indicated in the present claim,
the latter is then reacted according to the process
[A1] in an inert solvent with the cesium salt of an α-aminocarboxylic acid or an α-aminothiocarboxylic acid of the formula 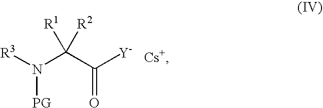
in which R¹, R² and R³ have the meaning indicated in claim 1,
PG is an amino protective group such as, for example, tert-butoxycarbonyl (Boc) or benzyloxycarbonyl (Z),
and
Y is O or S,
to give a compound of the formula 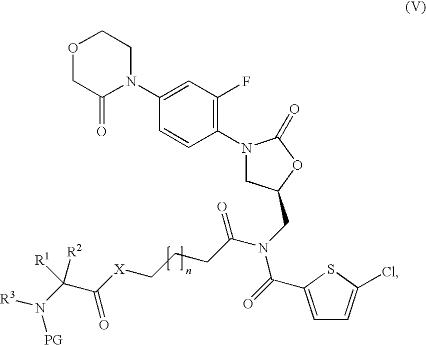
in which n, R¹, R² and R³ have the meaning indicated in claim 1,
PG has the meaning indicated in the present claim, and
X is O or S,
and subsequently the protective group PG is removed by conventional methods to result in a compound of the formula 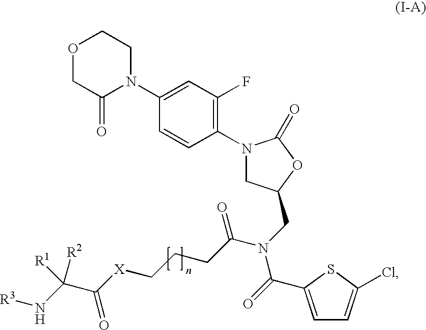
in which n, R¹, R² and R³ have the meaning indicated in claim 1,
X is O or S, or
[A2] is reacted in an inert solvent in the presence of a base with an α-aminothiocarboxylic acid of the formula 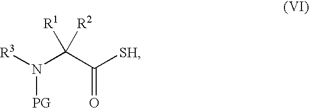
in which R¹, R² and R³ have the meaning indicated in claim 1,
PG is an amino protective group, such as, for example, tert-butoxycarbonyl (Boc) or benzyloxy-carbonyl (Z), to give a compound of the formula 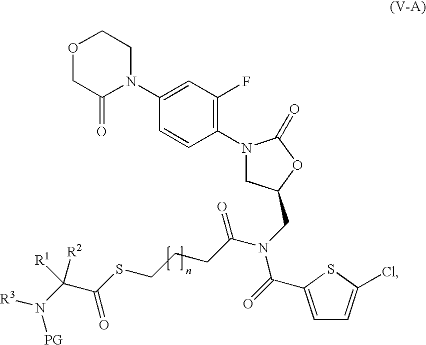
in which n, R¹, R² and R³ have the meaning indicated in claim 1, and
PG has the meaning indicated in the present claim, and subsequently the protective group PG is removed by conventional methods to result in a compound of the formula 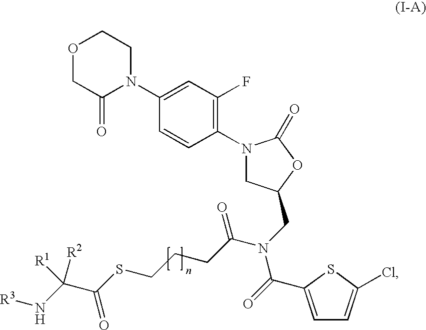
in which n, R¹, R² and R³ have the meaning indicated in claim 1,
or
[B] compound (A) is reacted in an inert solvent in the presence of a base with a compound of the formula 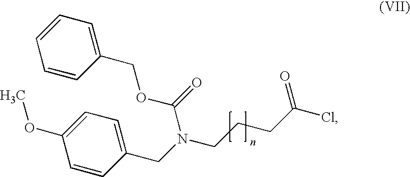
in which n has the meaning indicated in claim 1,
to give a compound of the formula 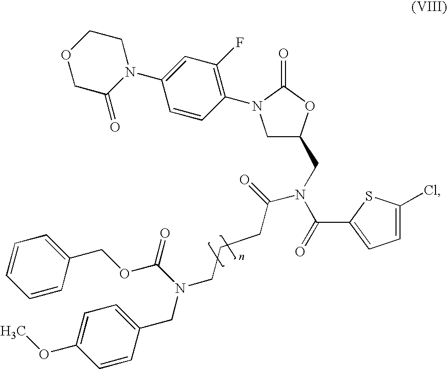
in which n has the meaning indicated in claim 1,
subsequently the protective groups are removed by conventional methods to result in a compound of the formula 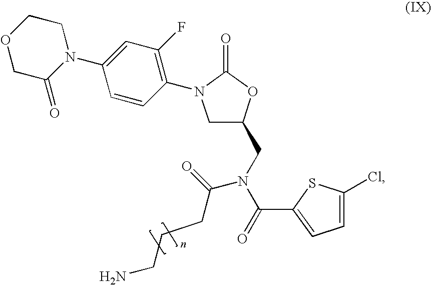
in which n has the meaning indicated in claim 1 and then, in the presence of a base, reacted with a compound of the formula 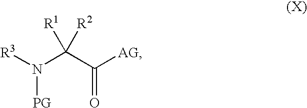
in which R¹, R² and R³ have the meaning indicated in claim 1,
AG is hydroxyl or halogen, preferably chlorine or bromine, or together with the carbonyl group forms an activated ester, preferably an N-hydroxysuccinimide ester, or a mixed anhydride, preferably an alkyl formate, particularly preferably an ethyl formate, and
PG is an amino protective group, such as, for example, tert-butoxycarbonyl (Boc) or benzyloxy-carbonyl (Z),
to give a compound of the formula 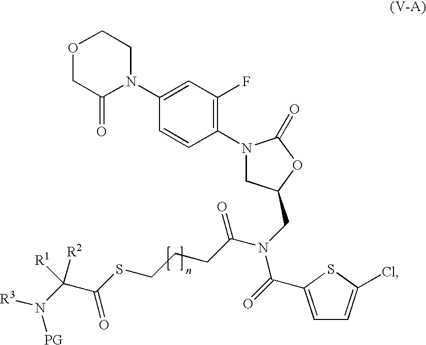
in which n, R¹, R² and R³ have the meaning indicated in claim 1, and
PG has the meaning indicated in the present claim,
and subsequently the protective group PG is removed by conventional methods to result in a compound of the formula 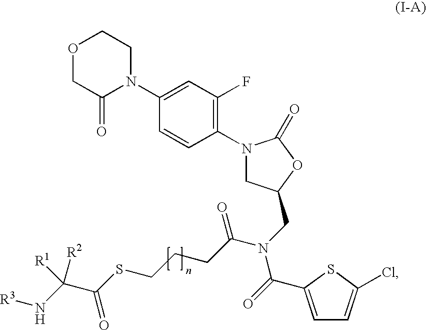
in which n, R¹, R² and R³ have the meaning indicated in claim 1,
and the compounds of the formula (I-A) or (I-B) resulting in each case are converted where appropriate into the salts thereof.

5-6. (canceled)

7. A medicament comprising a compound of the formula (I) as defined in claim 1 in combination with an inert, non-toxic, pharmaceutically suitable excipient.

8-9. (canceled)

10. A medicament as claimed in claim 7 adapted for intravenous use.

11. A method for the treatment and/or prophylaxis of thromboembolic disorders in humans and animals using at least one compound of the formula (I) as defined in claim 1.
